SEQUENCE LISTING (13 sequences)

SEQ ID NO: 1 - protein, 527 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 2 - protein, 240 aa, Homo sapiens
MLALLCSCLLLAAGASDAWTGEDSAEPNSDSAEWIRDMYAKVTEIWQEVMQRRDDDGALHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVGVCGPGLWERQAREHSERKKRRRESECKAA

SEQ ID NO: 3 - protein, 240 aa, Homo sapiens; identical to SEQ ID NO: 2

SEQ ID NO: 4 - protein, 240 aa, Homo sapiens; identical to SEQ ID NO: 2

SEQ ID NO: 5 - skipped

SEQ ID NO: 6 - protein, 527 aa, Homo sapiens; identical to SEQ ID NO: 1

SEQ ID NO: 7 - protein, 527 aa, Homo sapiens [sequence omitted]

SEQ ID NO: 8 - protein, 241 aa, Homo sapiens
MLALLCSCLLLAAGASDAWTGEDSAEPNSDSAEWIRDMYAKVTEIWQEVMQRRDDDGALHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASSLAGPHSIVGRAVVVHAGEDDLGRGGNQASVENGNAGRRLACCVVGVCGPGLWERQAREHSERKKRRRESECKAA

SEQ ID NO: 9 - DNA, 1518 bp, Homo sapiens
gctgacagccgggatcccgccagcgaccagatgcagcactggaaggagcagcgggccgcgcagaaagctgatgtcctgaccactggagctggtaacccagtaggagacaaacttaatgttattacagtagggccccgtgggccccttcttgttcaggatgtggttttcactgatgaaatggctcattttgaccgagagagaattcctgagagagttgtgcatgctaaaggagcaggggcctttggctactttgaggtcacacatgacattaccaaatactccaaggcaaaggtatttgagcatattggaaagaagactcccatcgcagttcggttctccactgttgctggagaatcgggttcagctgacacagttcgggaccctcgtgggtttgcagtgaaattttacacagaagatggtaactgggatctcgttggaaataacacccccattttcttcatcagggatcccatattgtttccatcttttatccacagccaaaagagaaatcctcagacacatctgaaggatccggacatggtctgggacttctggagcctacgtcctgagtctctgcatcaggtttctttcttgttcagtgatcgggggattccagatggacatcgccacatgaatggatatggatcacatactttcaagctggttaatgcaaatggggaggcagtttattgcaaattccattataagactgaccagggcatcaaaaacctttctgttgaagatgcggcgagactttcccaggaagatcctgactatggcatccgggatctttttaacgccattgccacaggaaagtacccctcctggactttttacatccaggtcatgacatttaatcaggcagaaacttttccatttaatccattcgatctcaccaaggtttggcctcacaaggactaccctctcatcccagttggtaaactggtcttaaaccggaatccagttaattactttgctgaggttgaacagatagccttcgacccaagcaacatgccacctggcattgaggccagtcctgacaaaatgcttcagggccgcctttttgcctatcctgacactcaccgccatcgcctgggacccaattatcttcatatacctgtgaactgtccctaccgtgctccaaattactaccccaacagctttggtgctccggaacaacagccttctgccctggagcacagcatccaatattctggagaagtgcggagattcaacactgccaatgatgataacgttactcaggtgcgggcattctatgtgaacgtgctgaatgaggaacagaggaaacgtctgtgtgagaacattgccggccacctgaaggatgcacaaattttcatccagaagaaagcggtcaagaacttcactgaggtccaccctgactacgggagccacatccaggctcttctggacaagtacaatgctgagaagcctaagaatgcgattcacacctttgtgcagtccggatctcacttggcggcaagggagaaggcaaatctg

SEQ ID NO: 10 - protein, 208 aa, Homo sapiens
MLALLCSCLLLAAGASDAWTGEDSAEPNSDSAEWIRDMYAKVTEIWQEVATLDAAQHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVG

SEQ ID NO: 11 - DNA, 624 bp, Homo sapiens
atgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcgccacgctggacgccgcgcagcacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggc

SEQ ID NO: 12 - protein, 538 aa, Homo sapiens
APRAATMPRVRSCLLHSPRTHALADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANLYPYDVPDYA

SEQ ID NO: 13 - DNA, 1569 bp, Homo sapiens
atgccacgcgtccgctcctgtcttctccacagtcccagaacacacgcactcgctgacagccgggatcccgccagcgaccagatgcagcactggaaggagcagcgggccgcgcagaaagctgatgtcctgaccactggagctggtaacccagtaggagacaaacttaatgttattacagtagggccccgtgggccccttcttgttcaggatgtggttttcactgatgaaatggctcattttgaccgagagagaattcctgagagagttgtgcatgctaaaggagcaggggcctttggctactttgaggtcacacatgacattaccaaatactccaaggcaaaggtatttgagcatattggaaagaagactcccatcgcagttcggttctccactgttgctggagaatcgggttcagctgacacagttcgggaccctcgtgggtttgcagtgaaattttacacagaagatggtaactgggatctcgttggaaataacacccccattttcttcatcagggatcccatattgtttccatcttttatccacagccaaaagagaaatcctcagacacatctgaaggatccggacatggtctgggacttctggagcctacgtcctgagtctctgcatcaggtttctttcttgttcagtgatcgggggattccagatggacatcgccacatgaatggatatggatcacatactttcaagctggttaatgcaaatggggaggcagtttattgcaaattccattataagactgaccagggcatcaaaaacctttctgttgaagatgcggcgagactttcccaggaagatcctgactatggcatccgggatctttttaacgccattgccacaggaaagtacccctcctggactttttacatccaggtcatgacatttaatcaggcagaaacttttccatttaatccattcgatctcaccaaggtttggcctcacaaggactaccctctcatcccagttggtaaactggtcttaaaccggaatccagttaattactttgctgaggttgaacagatagccttcgacccaagcaacatgccacctggcattgaggccagtcctgacaaaatgcttcagggccgcctttttgcctatcctgacactcaccgccatcgcctgggacccaattatcttcatatacctgtgaactgtccctaccgtgctccaaattactaccccaacagctttggtgctccggaacaacagccttctgccctggagcacagcatccaatattctggagaagtgcggagattcaacactgccaatgatgataacgttactcaggtgcgggcattctatgtgaacgtgctgaatgaggaacagaggaaacgtctgtgtgagaacattgccggccacctgaaggatgcacaaattttcatccagaagaaagcggtcaagaacttcactgaggtccaccctgactacgggagccacatccaggctcttctggacaagtacaatgctgagaagcctaagaatgcgattcacacctttgtgcagtccggatctcacttggcggcaagggagaaggcaaatctg

(19) United States
(12) Patent Application Publication
(10) Pub. No.: US 2021/0381002 A1
(43) Pub. Date: Dec. 9, 2021

(54) GENE THERAPY FOR OXIDATIVE STRESS

(71) Applicant: Cornell University, Ithaca, NY (US)

(72) Inventors: Ronald G. Crystal, New York, NY (US); Stephen M. Kaminsky, Bronx, NY (US); Christiana Salami, Bronx, NY (US)

(21) Appl. No.: 17/045,110

(22) PCT Filed: Apr. 3, 2019
(86) PCT No.: PCT/US2019/025529

§ 371 Date: Oct. 2, 2020

Related U.S. Application Data
(60) Provisional application No. 62/652,098, filed on Apr. 3, 2018.

(51) Int. Cl. C12N 15/86; A61K 48/00; C12N 9/08; C12N 9/02; A61P 39/06; A61P 11/00

(52) U.S. Cl. CPC C12N 15/86; A61K 48/005; C12N 9/0065; C12Y 111/01006; C12N 2750/14171; C12Y 115/01001; A61P 39/06; A61P 11/00; C12N 2750/14143; C12N 9/0089

(57) ABSTRACT

Compositions and methods for anti-oxidant therapy are provided.

### CROSS-REFERENCE TO RELATED APPLICATIONS

This application claims the benefit to the filing date of U.S. application No. 62/652,098, filed on Apr. 3, 2018, the disclosure of which is incorporated by reference herein.

### BACKGROUND

Oxidative stress, a common cause of tissue damage with concomitant initiation or acceleration of disease, is a known risk factor for atherosclerosis, chronic lung disorders such as chronic obstructive pulmonary disease and fibrotic lung disorders, cancer, diabetes, rheumatoid arthritis, post-ischemic perfusion injury, myocardial infarction, cardiovascular diseases, chronic inflammation, stroke and septic shock, aging and other degenerative and neurological diseases such as Alzheimer's and Parkinson's, and contributes to the aging process (Durackova, 2010; Mitscher et al., 1996).

Oxidants derive from environmental sources including industrial pollution, cosmic radiation and cigarette smoke or from normal cellular function such as respiratory burst from neutrophils and monocytes as well as detoxification enzymes. Oxidative stress is mediated by free radicals that include hydroxyl and superoxide, which in turn lead to reactive species such as hydrogen peroxide, together referred to as reactive oxygen species (ROS). These oxidants damage lipids, proteins and DNA, which mediate numerous pathogenic outcomes described above and numerous studies have demonstrated that antioxidant compounds can be protective for atherosclerosis, cancer, mutagenesis and inflammation (Mitscher et al., 1996; Uttara et al., 2009; Owen et al., 2000; Sala et al., 2002).

For natural protection to oxidative stress, the antioxidant proteins catalase and superoxide dismutase catalyze the neutralization of hydrogen peroxide and superoxide, respectively (Mates et al., 1999; Birben et al., 2012). These enzymes represent a line of defense to oxidative stress initiated by normal cellular process in healthy individuals but do not have the capacity to address the excessive burden of oxidants derived from environmental or hyper-disease states due to magnitude or physiological localization. For example, catalase is a tetrameric intracellular protein and therefore is not found in sera nor on the mucosal surfaces where it can act as a first line of defense to exogenous ROS (Coyal et al., 2010). SOD has three forms, SOD1, SOD2, and SOD3, which are defined by their cellular locations, cytoplasm, mitochondria and extracellular space bound to heparin, respectively (Perry et al., 2010). Similar to catalase, the SOD enzymes are not present in the sera or mucosal surface. SOD3 is secreted, but it has a heparin-binding domain that attaches to the cell surface and thus is not available in sufficient levels unbound to the cell surface to perfuse across the epithelial surface of organs and reach mucosal surfaces (Perry et al., 2010).

### SUMMARY

A gene therapy approach is provided that mediates expression of secreted anti-oxidant enzymes providing protection against pathogenic extracellular oxidative stress, including at mucosal surfaces. Long-term expression by adeno-associated virus, retrovirus or lentivirus vector, constructed with a cDNA that encodes a monomeric secreted functional catalase and a modified extraceilular superoxide dismutase, both to provide a frontline defense to exogenous or inappropriate levels of reactive oxygen species. To address this, the sequences of both catalase and SOD3 were modified to facilitate secretion and diffusion and specifically for SOD to remain unattached to the cell surface. The genetic code for these secreted forms of catalase and SOD are incorporated into, in one embodiment, an adeno associated viral vector, to provide persistent and consistent levels of these anti-oxidant enzymes in a treated region, in the sera, and across epithelial and mucosal surfaces as a barrier to environmental and pathogenic assaults by the oxidant species hydrogen peroxide and superoxide. The strategy for constructing a secreted monomeric catalase removes a relatively non-structured region that mediates the inter-molecular adherence required for tetramer formation and the addition of a secretion signaling sequence. SOD3 was modified to replace a loop that engages monomers to form a larger tetramer with a segment in the SOD3 sequence and the heparin binding domain was removing the capacity to adhere to the extracellular matrix. Both modified catalase and SOD3 were shown to be released into the sera and maintain function. In one embodiment, the two anti-oxidants enzymes may be delivered in separate vectors. In one embodiment, the vectors may be any serotype of AAV vector. In one embodiment, the vector may be a plasmid vector or other viral vector such as a retrovirus, adenovirus or lentivirus vector. Any expression cassette may be used and different alterations to the protein sequences for catalase and SOD3 may be made.

The administration of the vector may result in protection against environmentally derived oxidative stress insult, e.g., as a result of radiation or chemical exposure such as in a nuclear attack or a gas (terror) attack, cigarette or other tobacco use including E-cigarettes or vaping, or cigar smoke exposure, or inappropriate endogenous ROS from inflammatory responses. In one embodiment, the vector may be used in a method to prevent, inhibit or treat one or more disorders including but not limited to atherosclerosis, cancer, diabetes, rheumatoid arthritis, post-ischemic perfusion injury, myocardial infarction, cardiovascular diseases, chronic inflammation, stroke and septic shock, aging and other degenerative and neurological diseases such as Alzheimer's and Parkinson's and contribute to the aging process.

In one embodiment, a gene therapy vector comprising an expression cassette comprising a nucleic acid sequence coding for a modified catalase that has catalase activity but does not form tetramers is provided. In one embodiment, the catalase has at least 80%, 82%, 84%, 85%, 87%, 90%, 92%, 94%, 95%, 96% 98%, 99% or more amino acid sequence identity to one of SEQ ID Nos. 1, 5-7 or 12. In one embodiment, the gene therapy vector further comprises a nucleic acid sequence coding for a modified superoxide dismutase that is secreted but does not bind to cell surfaces and optionally does not form tetramers. In one embodiment, the superoxide dismutase has at least 80%, 82%, 84%, 85%, 87%, 90%, 92%, 94%, 95%, 96% 98%, 99% or more amino acid sequence identity to one of SEQ ID Nos. 2-4, 8 or 10. In one embodiment, a gene therapy vector comprising an expression cassette comprising a nucleic acid sequence coding for a modified superoxide dismutase that is secreted but does not bind to cell surfaces is provided. In one embodiment, the modified superoxide dismutase is a modified superoxide dismutase-3. In one embodiment, the modified superoxide dismutase does not bind to heparin. In one embodiment, the modified superoxide dismutase does not form tetramers. In one embodiment, the modified catalase has a deletion in the N-terminus in the threading arm domain, which deletion may be of 1 to 80 or more residues or any integer between 1 and 80, for example, a deletion of 5, 10, 15, 16, 17, 18, 19, 20, 21, 22, 23, 24, 25, 30, 35, 40, 45, 50, 55, 60, 65, 70 or 75 residues. In one embodiment, the modified catalase has a deletion in the N-terminus, for example, a deletion of 5, 10, 15, 16, 17, 18, 19, 20, 21, 22, 23, 24, or 25 residues. In one embodiment, the modified catalase has a deletion in the N-terminus, for example, a deletion of 15 or 20 or 20 to 25 residues. In one embodiment, the modified catalase has a deletion in the wrapping loop domain, which deletion may be 1, 2, 3, 4, 5, 6, 7, 8, 9, 10, 11, 12, 13, 14, 15, 16, 17, 18, 19, 20, 21, 22, 23, 24, or 25 residues. In one embodiment, the modified catalase has a deletion in the wrapping loop domain, which deletion may be 15 to 20 or 20 to 25 residues. In one embodiment, the modified catalase has a deletion in the wrapping loop domain, which deletion may be from position 379, 380, 381, 382 383, 384 or 385 to about position 398, 399, 400, 401, 402 or 403, e.g., position 381 to 400 in catalase (see FIG. 2). In one embodiment, the modified catalase has a deletion in the threading arm and the wrapping loop domains. In one embodiment, the modified catalase has a secretory sequence, e.g., a heterologous secretory sequence. In one embodiment, the modified superoxide dismutase has a deletion in the heparin binding domain, which deletion in the loop residues may be of 1 to 15 or 20 or 25 or more residues or any integer between 1 and 15, for example, a deletion of 2, 3, 4, 5, 6, 7, 8, 9, 10, 11, 12, 13, 14, 15, 20, 23 or 25 residues. In one embodiment, the modified superoxide dismutase has a replacement of one or more residues of a turn or loop domain, for example, a replacement of 1, 2, 3, 4, 5, 6, 7, 8, 9, 10, 11, 12, 13, 14, 15, 20, 23, 25, or more residues with 1, 2, 3, 4, 5, 6, 7, 8, 9, 10, 11, 12, 13, 14, 15, 20, 23, 25, or more residues. In one embodiment, the modified superoxide dismutase has a replacement of one or more residues of a turn or loop domain, for example, a replacement of 6, 7, 8, 9, 10, 11, or 12 residues with 4, 5, 6, 7, 8, 9, or 10 residues. In one embodiment, the modified superoxide dismutase has a replacement of residues of a turn or loop domain, for example, at position 46, 47, 48, 49, 50, 51 52, or 53 to position 56, 57, 58, 59, 60, 61 or 62 with residues at position 66, 67, 68, 69, 70, or 71 to position 72, 73, 74, 75, 76, 77, 78 or 79, or position 70, 71, 72, 73, 74, or 75 to position 77, 78, 79, 80, 81, 82 or 83 in superoxide dismutase (see FIG. 3). In one embodiment, the modified superoxide dismutase has a deletion and an insertion of one or more residues of a turn or loop domain, for example, a deletion of 1, 2, 3, 4, 5, 6, 7, 8, 9, 10, 11, 12, 13, 14, 15, 20, 23, 25, or more residues. In one embodiment, the modified superoxide dismutase has a deletion of one or more residues of a heparin binding domain, for example, a replacement of 1, 2, 3, 4, 5, 6, 7, 8, 9, 10, 11, 12, 13, 14, 15, 20, 23, 25, or more residues. In one embodiment, the modified superoxide dismutase has a deletion of one or more residues of a heparin binding domain, for example, a deletion of 10 to 30, e.g., 18, 19, 20, 21 or 22 residues, or a deletion of 25, 26, 27, 28, 29 or 30 residues. In one embodiment, the modified superoxide dismutase has a deletion of a heparin binding domain, for example, from position 209, 210, 211, 212, 213, 214 or 215 to position 235, 236, 237, 238, 239, or 240, or from position 217, 218, 219, 220, 221, 222, or 223 to position 235, 236, 237, 238, 239, or 240 in superoxide dismutase.

In one embodiment, the gene therapy vector is a viral vector, e.g., an adenovirus, adeno-associated virus (AAV), retrovirus or lentivirus vector. In one embodiment, the AAV vector is AAV2, AAV5, AAV7, AAV8, AAV9 or AAVrh.10. In one embodiment, one vector comprises an expression cassette comprising a nucleic acid sequence coding for the modified catalase and the modified superoxide dismutase, which catalase sequence and superoxide dismutase sequence are separated by a protease substrate sequence. In one embodiment, the modified catalase is N-terminal to the modified superoxide dismutase. In one embodiment, the modified catalase is C-terminal to the modified superoxide dismutase. In one embodiment, one vector comprises an expression cassette comprising a nucleic acid sequence coding for the modified catalase and another vector comprises an expression cassette comprising a nucleic acid sequence coding for the modified superoxide dismutase.

Also provided is a pharmaceutical composition comprising an amount of the vector. In one embodiment, the vector is on a plasmid. In one embodiment, the vector is a viral vector, e.g., an adenovirus, adeno-associated virus (AAV), retrovirus or lentivirus vector. In one embodiment, the AAV vector is pseudotyped. In one embodiment, the AAV vector is pseudotyped with AAVrh.10, AAV8, AAV9, AAV5, AAVhu.37. AAVhu.20, AAVhu.43, AAVhu.8, AAVhu.2, or AAV7 capsid. In one embodiment,

the AAV genome of the vector is AAV2, AAV5, AAV7. AAV8, AAV9 or AAVrh.10. In one embodiment, the amount of the vector is about 1×10¹¹ to about 1×10¹⁶ genome copies. In one embodiment, the amount of the vector is about 1×10¹² to about 1×10¹⁵ genome copies, about 1×10¹¹ to about 1×10¹³ genome copies, or about 1×10¹³ to about 1×10¹⁵ genome copies. In one embodiment, the pharmaceutical composition further comprises a pharmaceutically acceptable carrier. In one embodiment, the pharmaceutical composition comprises a viral vector encoding the modified catalase and another viral vector encoding the modified superoxide dismutase.

Also provided is a method to prevent, inhibit or treat oxidative damage in a mammal, comprising: administering to the mammal, an effective amount of the vector or the pharmaceutical composition. In one embodiment, the mammal has or is at risk of having atherosclerosis, cancer, diabetes, rheumatoid arthritis, post-ischemic perfusion injury, myocardial infarction, cardiovascular diseases, chronic inflammation, stroke, septic shock, or other degenerative and neurological diseases such as Alzheimer's disease or Parkinson's disease. In one embodiment, the mammal is a human. In one embodiment, an amount of a viral vector encoding the modified catalase and an amount of the viral vector encoding the modified superoxide dismutase is administered. In one embodiment, the viral vectors are administered sequentially. In one embodiment, the viral vectors are administered concurrently. In one embodiment, a viral vector encoding the modified catalase and the modified superoxide dismutase is administered. In one embodiment, the AAV vector is AAVrh.10, AAV8, AAV9. AAV5. AAVhu.37. AAVhu.20, AAVhu.43, AAVhu.8, AAVhu.2, or AAV7 capsid. In one embodiment, the AAV vector is AAVrh.10, AAV8, or AAV5. In one embodiment, the AAV vector is AAV2. AAV5, AAV7, AAV8. AAV9 or AAVrh.10. A dose of the viral vector may be about 1×10¹¹ to about 1×10¹⁶ genome copies, about 1×10¹² to about 1×10¹⁵ genome copies about 1×10¹¹ to about 1×10¹³ genome copies, or about 1×10¹³ to about 1×10¹⁵ genome copies. In one embodiment, the AAV vector is pseudotyped with AAVrh.10, AAV8, AAV9, AAV5, AAVhu.37, AAVhu.20, AAVhu.43. AAVhu.8. AAVhu.2, or AAV7 capsid. In one embodiment, the AAV vector is pseudotyped with AAVrh.10. AAV8, or AAV5. In one embodiment, the AAV vector is AAV2, AAV5. AAV7, AAV8, AAV9 or AAVrh.10.

Further provided is a method to prevent, inhibit or treat COPD, respiratory distress syndrome or fibrotic interstitial lung disease in a mammal, comprising: administering to a mammal in need thereof, an effective amount of the vector or the pharmaceutical composition. In one embodiment, the mammal is a human. In one embodiment, an amount of a viral vector encoding the modified catalase and an amount of the viral vector encoding the modified superoxide dismutase is administered. In one embodiment, the viral vectors are administered sequentially. In one embodiment, the viral vectors are administered concurrently. In one embodiment, a viral vector encoding the modified catalase and the modified superoxide dismutase is administered. In one embodiment, the AAV vector is AAVrh.10, AAV8, AAV9, AAV5, AAVhu.37, AAVhu.20, AAVhu.43, AAVhu.8, AAVhu.2, or AAV7 capsid. In one embodiment, the AAV vector is AAVrh.10, AAV8, or AAV5. In one embodiment, the AAV vector is AAV2, AAV5, AAV7, AAV8, AAV9 or AAVrh.10. A dose of the viral vector may be about 1×10¹¹ to about 1×10¹⁶ genome copies, about 1×10¹² to about 1×10¹⁵ genome copies about 1×10¹¹ to about 1×10¹³ genome copies, or about 1×10¹³ to about 1×10¹⁵ genome copies. In one embodiment, the AAV vector is pseudotyped with AAVrh.10, AAV8, AAV9, AAV5, AAVhu.37, AAVhu.20, AAVhu.43, AAVhu.8. AAVhu.2, or AAV7 capsid. In one embodiment, the AAV vector is pseudotyped with AAVrh.10, AAV8, or AAV5. In one embodiment, the AAV vector is AAV2, AAV5, AAV7, AAV8, AAV9 or AAVrh.10.

### BRIEF DESCRIPTION OF THE DRAWINGS

FIGS. 1A-B. Oxidant burden and anti-oxidant defenses of the lung. A) The lung is stressed with inhaled oxidants and in COPD, with endogenous extracellular oxidants from activated inflammatory cells. B) Lung cells have 3 major enzyme antioxidant defenses: SOD (catalyzes O₂⁺ to H₂O₂; catalase (H₂O₂ to H₂O) and glutathione (GSH; H₂O₂ to H₂O; oxidized GSH forms GSSG which is reduced by multiple intracellular enzymes). None of these enzyme systems provide adequate extracellular antioxidant defenses.

FIGS. 2A-B. Modifications of catalase to create a functional, extracellular monomer. A) Human catalase monomer structure. B) Human catalase monomer amino acid sequence (SEQ ID NO:1). Shown are the regions where modifications made to the N-terminus and wrapping loop domain. Three strategies were used: hCatNT⁻ (20 amino acid deletion from the N-terminus), hCatWL⁻ (20 amino acid deletion from the wrapping loop domain) and hCat⁻NT⁻WL⁻ (combination of the 2 deletions). To direct secretion, a 5′ signal peptide was added; to detect the protein, a 3′ hemagglutinin (HA) tag was added.

FIGS. 3A-B. Modifications to generate a functional, extracellular SOD3 monomer. A) SOD3 monomer structure. B) Human SOD3 amino acid sequence (SEQ ID NO:8). Shown are the regions where modifications were made with details of the modifications.

FIG. 4. LEX 5, a gene therapy-based, extracellular, diffusable enzymatic antioxidant, vector was 1 of 4 candidate gene transfer vectors (LEX 5a, b, c and d). All candidates have the identical expression cassette, differing only in the antioxidant enzyme coding sequences. All are packaged in the AAVrh.10 capsid to generate the 4 candidate vectors.

FIGS. 5A-C. Assessment of the modified catalase constructs. A) Secretion of the modified catalase constructs. Western (SDS reducing gel; anti-HA tag) of supernatant of 293T cells transfected with the 3 modified catalase constructs. Lane 1—mock; lane 2—hCatNT⁻ construct; lane 3—hCATWL⁻; lane 4—hCATNT-WL⁻; and lane 5—catalase control. B) Analysis of catalase activity of the supernatants generated by the 3 constructs. All 3 are secreted, but only hCatWL⁻ was active. C) Analysis of the supernatants from the hCatWD⁻ construct in Bis Tris gels to demonstrate that the hCatND⁻ construct is monomeric.

FIGS. 6A-B. Modified SOD3 construct. A) Secretion of the modified SOD3 constructs. Western (anti-HA tag) of supernatant of 293T cells transfected with: lane 1—mock; lane 2—unmodified HA tagged SOD3; lane 3—SOD3hd⁻. Only SOD3hd⁻ is in the supernatant. B) Anti-HA Western of fractions from the SOD3hd⁻ supernatants run on a Sephacryl sizing column construct to demonstrate that the SOD3hd-construct is monomeric. Approximate MW are calculated from column specifications.

FIGS. 7A-F. Assessment of the function of LEX 5a and LEX 5b in vivo. AAVrh.10hCatWD⁻ (LEX 5a) or AAVrh.10hSOD3hd⁻ (LEX 5b) were administered intravenously to male Balb/c mice (10¹¹ genome copies total dose). Two wk later, liver and lung were sampled for vector DNA and serum was assessed for catalase and SOD activity. A-C) LEX 5a. A) Liver vector DNA; B) Lung vector DNA; and C) Serum catalase activity. D-F) LEX 5b. D) Liver vector DNA; E) Lung vector DNA; and F) Serum SOD activity.

FIG. 8. Catalase structure. Each monomer has four domains. In the first domain, the amino-terminal residues include those for the interlocking arm exchange that binds the monomeric units together. The second domain is the heme domain. The third domain is the wrapping loop domain, where the four monomers wrap around each other to form a tetramer, salt bridges and ionic interactions between positively and negatively charged amino acid side chains hold the four monomers together. The fourth domain includes carboxy-terminal residues that play a role in orienting the incoming H₂O₂ substrate for catalytic degradation.

FIG. 9. Genetic modifications to inhibit catalase tetramer formation. To prevent monomer formation, amino acid residues in the N-terminus threading arm and/or wrapping loop domains are deleted while maintaining the reading frame and ensure that NADPH binding and enzymatic functions are not modified.

FIG. 10. Modified catalase sequences.

FIG. 11. In vitro characterization of the tetramer catalase. Despite the secretion signal, the wild-type catalase stays in the cells (the Western analysis breaks up the tetramer to monomeric units for analysis).

FIG. 12. In vitro characterization of modified catalase constructs.

FIGS. 13A-B. In vitro characterization of modified catalase constructs. A) Western blot. B) Catalase activity in supernatant.

FIG. 14. Assessment of supernatants of catalase constructs for monomeric and multimeric catalase separation. Samples were assessed on Bis-Tris gel, blotted and probed with anti-catalase antibody (ABCAM: ab88067); predicted band sizes, 60 kDa for monomer and 240 kDa for tetramer, aAll 3 constructs secreted only the monomeric construct.

FIG. 15. In vitro assessment of supernatants for catalase activity by constructs for monomeric and multimeric catalase. Monomeric constructs express proteins with catalase activity.

FIGS. 16A-B. In vivo assessment of human catalase expression by an AAVrh.10 coding for a human catalase with a modified wrapping domain (hCatWL⁻). A) Experimental design. B) Vector copy number in the liver.

FIGS. 17A-B. Long-term time course of in vivo catalase activity mediated by an adeno-associated virus serotype rh.10 coding for a human catalase with a modified wrapping loop domain (hCatWL⁻). A) Vector design. B) Experimental design.

FIG. 18. Long-term time course of in vivo catalase activity mediated by an adeno-associated virus serotype rh.10 coding for human catalase wrapping loop domain modified (hCatWL⁻). Catalase activity in serum of male C57bl/6J mice from the following treatment groups from week 2 to week 12. PBS (n=4). AAVrh.10hCATWL⁻ (n=5 up to week 4, 1 mouse was sacrificed at week 4 and then another mouse was sacrificed at week 8). hCatWL⁻ (20 AA deletion from wrapping loop domain and HA tag).

FIGS. 19A-B. Superoxide dismutase 3. A) Protein structure. B) Crystallographic structure.

FIG. 20. Modified SOD3 to enhance extracellular availability. To enhance extracellular diffusion, the “turn” residues in the heparin-binding domain are modified.

FIG. 21. In vitro assessment of hSOD3hd⁻.

FIG. 22. Analysis of monomeric and multimeric forms of SOD3 in supernatant expressed by SOD3hd⁻. Fractions from a size exclusion column were run on Bis-Tris gel. Western assay with anti-HA antibody; monomer MW 30 kDa is expected in fractions for lanes 2-5.

FIGS. 23A-B. SOD3hd⁻ gDNA quantification in liver and lung following IV administration. A) DNA quantification in liver. B) DNA quantification in lung.

FIG. 24. Modified SOD3 activity in serum (Wk 2).

FIG. 25. In vitro antioxidant protection mediated by modified SOD3 and catalase of oxidant exposed large airway epithelial cells. The data shows the antioxidant properties of the modified SOD3 and catalase to protect human large airway epithelial cells from the oxidants derived from xanthine oxidase and cigarette smoke extract (CSE).

FIGS. 26A-B. In vitro antioxidant protection mediated by modified SOD3 and catalase of oxidant exposed large airway epithelial cells. LDH assay measures cell death, and lower LDH indicates protection from oxidant-induced cell death. Modified SOD3 provided enhanced protection against cigarette smoke extract and xanthine oxidase derived oxidants (reduced LDH activity from CSE or xanthine oxidase exposure). Both modified SOD3 and modified catalase provide better protection against xanthine oxidase exposure compared to the unmodified SOD3 and catalase.

FIG. 27. Schematic of two constructs.

### DETAILED DESCRIPTION

In the following description, reference is made to the accompanying drawings that form a part hereof, and in which is shown by way of illustration specific embodiments which may be practiced. These embodiments are described in detail to enable those skilled in the art to practice the invention, and it is to be understood that other embodiments may be utilized and that logical changes may be made without departing from the scope of the present invention. The following description of example embodiments is, therefore, not to be taken in a limited sense, and the scope of the present invention is defined by the appended claims.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by heterologousization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment described herein that is a polynucleotide encompasses both the double-stranded form and each of two complementary single-stranded forms known or predicted to make up the double-stranded form.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this invention are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette.” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphonylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less e.g., with 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or with 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic; trp, tyr, phe.

The invention also envisions polypeptides with non-conservative substitutions. Non-conservative substitutions entail exchanging a member of one of the classes described above for another.

### Compositions and Methods

As a mechanism to address oxidative stress there is no precedent to the modifications described herein to two antioxidant enzymes used individually or in combination in a gene transfer approach. In one embodiment, protein modifications were designed and genetic constructs prepared for secreted monomeric forms of the catalase and superoxide dismutase 3 enzymes. The incorporation of the genetic code for each was inserted into the expression cassette of, for example a virus vector such as an adeno-associated viral vector, either separately, or combined as a single translated sequence with an intervening cleavage site. The vector-mediated expression provides either catalase or SOD or both to the extracellular milieu, with transfer to the sera and mucosal surfaces as a frontline barrier to oxidative stress. The use of these gene transfer approaches protects against oxidation-mediated pathology and disease.

The use of gene therapy is based on a persistent expression vector such as an adeno-associated virus (AAV) vector (but could be another viral vector such as a retro or lenti virus vector). The 3-dimensional protein structures of human catalase and human SOD3 enzymes, which are the potent anti-oxidant weapons for protection against ROS, were examined. A strategy for modifying each enzyme with the goal of vector-mediated endogenous production of secreted, monomeric, functional constructs that can provide a front line defense to perfuse across the epithelial surface of organs and reach mucosal surfaces, was devised.

Catalase. In one embodiment, an addition to the genetic code at the N terminus of the protein of a secretion signal peptide (e.g., from human immunoglobulin; see SEQ ID NO:13) to provide instructions to the cell protein production machinery to direct the translated sequence to be secreted from the cell. In one embodiment, the genetic code was modified to remove sequences that encode a loop in the protein sequence that forms the binding interface between monomers in the tetramer. In one embodiment, the DNA encoding amino acids 381 to 400 were deleted in such a way that the ends in the 3-dimensional protein structure remained close to one another and therefore the trimmed protein chain is not constrained by the removal of the intervening loop to minimize impact on the overall protein structure.

SOD3. In one embodiment, the genetic code for amino acid residues 50 to 59 were removed and replaced with that for amino acids 74 to 80, another flexible loop in the SOD3 structure chosen to minimize immunity that could occur by the introduction of non-SOD3 sequences into the structure. In one embodiment, a deletion from the genetic code for the region that encodes the extracellular matrix/heparin binding domain (amino acids 220 to 240) to enable the secreted protein to freely diffuse from the cell surface. Because wild type SOD3 encodes a secretion signal sequence no changes were made to cDNA encoding the amino terminus.

In one embodiment, both transgenes were placed into an expression cassette behind the constitutive expressing cytomegalovirus (CMV)/chicken beta-actin hybrid promoter and incorporated in the AAVrh.10 serotype vector. An intervening furin-2a cleavage sequence provides the capacity for the single translated sequence to produce two separate polypeptide products, secreted monomer catalase and secreted monomer SOD3 (Fang et al., 2005).

Exemplary amino acid sequences for catalase are provided in SEQ ID NO:1 and SEQ ID Nos. 5-7 or 12, and exemplary SOD sequences are provided in SEQ ID Nos. 2-4, 8 and 10:

| SEQ ID NO: 2 | |
|---|---|
| 1 | mlallcscll laagasdawt gedsaepnsd saewirdmya kvteiwqevm qrrdddgalh |
| 61 | aacqvqpsat ldaaqprvtg vvlfrqlapr akldaffale gfptepnsss raihvhqfgd |
| 121 | lsqgcestgp hynplavphp qhpgdfgnfa vrdgslwryr aglaaslagp hsivgravvv |
| 181 | hageddlgrg gnqasvengn agrrlaccvv gvcgpglwer qarehserkk rrreseckaa |
| SEQ ID NO: 3 | |
| 1 | mlallcscll laagasdawt gedsaepnsd saewirdmya kvteiwqevm qrrdddgalh |
| 61 | aacqvqpsat ldaaqprvtg vvlfrqlapr akldaffale gfptepnsss raihvhqfgd |
| 121 | lsqgcestgp hynplavphp qhpgdfgnfa vrdgslwryr aglaaslagp hsivgravvv |
| 181 | hageddlgrg gnqasvengn agrrlaccvv gvcgpglwer qarehserkk rrreseckaa |
| SEQ ID NO: 4 | |
| 1 | mlallcscll laagasdawt gedsaepnsd saewirdmya kvteiwqevm qrrdddgalh |
| 61 | aacqvqpsat ldaaqprvtg vvlfrqlapr akldaffale gfptepnsss raihvhqfgd |
| 121 | lsqgcestgp hynplavphp qhpgdfgnfa vrdgslwryr aglaaslagp hsivgravvv |
| 181 | hageddlgrg gnqasvengn agrrlaccvv gvcgpglwer qarehserkk rrreseckaa |
| SEQ ID NO: 6 | |
| 1 | madsrdpasd qmqhwkeqra aqkadvlttg agnpvgdkln vitvgprgpl lvqdvvftde |
| 61 | mahfdrerip ervvhakgag afgyfevthd itkyskakvf ehigkktpia vrfstvages |
| 121 | gsadtvrdpr gfavkfyted gnwdlvgnnt piffirdpil fpsfihsqkr npqthlkdpd |
| 181 | mvwdfwslrp eslhqvsflf sdrgipdghr hmngygshtf klvnangeav yckfhyktdq |
| 241 | giknlsveda arlsqedpdy girdlfnaia tgkypswtfy iqvmtfnqae tfpfnpfdlt |
| 301 | kvwphkdypl ipvgklvlnr npvnyfaeve qiafdpsnmp pgieaspdkm lqgrlfaypd |
| 361 | thrhrlgpny lhipvncpyr arvanyqrdg pmcmqdnqgg apnyypnsfg apeqqpsale |
| 421 | hsiqysgevr rfntanddnv tqvrafyvnv lneeqrkrlc eniaghlkda qifiqkkavk |
| 481 | nftevhpdyg shiqalldky naekpknaih tfvqsgshla arekanl |
| SEQ ID NO: 7 | |
| 1 | madsrdpasd qmqhwkeqra aqkadvlttg agnpvgdkln vitvgprgpl lvqnvvftde |
| 61 | mahfdrerip ervvhakgag afgyfevthd itkyskakvf ehigkktpia vrfstvages |
| 121 | gsadtvrdpr gfavkfyted gnwdlvgnnt piffirdpil fpsfihsqkr npqthlkdpd |
| 181 | mvwdfwslrp eslhqvsflf sdrgipdghr hmngygshtf klvnangeav yckfhyktgq |
| 241 | giknlsveda arlsqedpdy girdlfnaia tgkdpswtfy iqvmtfnqae tfpfnpfdlt |
| 301 | rvwphkdypl ipvgklvlnr npvnyfaeve qiafdpsnmp pgieaspdkm lqgrlfaypd |
| 361 | thrhrlgpny lhipvncpyr arvanyqrdg pmcmqdnqgg apnyypnsfg apeqqpsale |
| 421 | hsiqysgevr rfntanddnv tqvrafyvnv lneeqrkrlc eniaghlkda qifiqkkavk |
| 481 | nftevhpdyg shiqalldky naekpknaih tfvrsgshlv arekanl |

(SEQ ID NO: 9; hCatWL⁻)
GCTGACAGCCGGGATCCCGCCAGCGACCAGATGCAGCACTGGAAGGAGC
AGCGGGCCGCGCAGAAAGCTGATGTCCTGACCACTGGAGCTGGTAACCCA
GTAGGAGACAAACTTAATGTTATTACAGTAGGGCCCCGTGGGCCCCTTCT
TGTTCAGGATGTGGTTTTCACTGATGAAATGGCTCATTTTGACCGAGAGAG
AATTCCTGAGAGAGTTGTGCATGCTAAAGGAGCAGGGGCCTTTGGCTACT
TTGAGGTCACACATGACATTACCAAATACTCGAAGGCAAAGGTATTTGAG
CATATTGGAAAGAAGACTCCCATCGCAGTTCGGTTCTCCACTGTTGCTGGA
GAATCGGGTTCAGCTGACACAGTTCGGGACCCTCGTGGGTTTGCAGTGAA
ATTTTACACAGAAGATGGTAACTGGGATCTCGTTGGAAATAACACCCCCA
TTTTCTTCATCAGGGATCCCATATTGTTTCCATCTTTTATCCACAGCCAAAA
GAGAAATCCTCAGACACATCTGAAGGATCCGGACATGGTCTGGGACTTCT
GGAGCCTACGTCCTGAGTCTCTGCATCAGGTTTCTTTCTTGTTCAGTGATC
GGGGGATTCCAGATGGACATCGCCACATGAATGGATATGGATCACATACT
TTCAAGCTGGTTAATGCAAATGGGGAGGCAGTTTATTGCAAATTCCATTAT
AAGACTGACCAGGGCATCAAAAACCTTTCTGTTGAAGATGCGGCGAGACT
TTCCCAGGAAGATCCTGACTATGGCATCCGGGATCTTTTTAACGCCATTGC
CACAGGAAAGTACCCCTCCTGGACTTTTTACATCCAGGTCATGACATTTAA
TCAGGCAGAAACTTTTCCATTTAATCCATTCGATCTCACCAAGGTTTGGCC
TCACAAGGACTACCCTCTCATCCCAGTTGGTAAACTGGTCTTAAACCGGA
ATCCAGTTAATTACTTTGCTGAGGTTGAACAGATAGCCTTCGACCCAAGC
AACATGCCACCTGGCATTGAGGCCAGTCCTGACAAAATGCTTCAGGGCCG
CCTTTTTGCCTATCCTGACACTCACCGCCATCGCCTGGGACCCAATTATCT
TCATATACCTGTGAACTGTCCCTACCGTGCTCCAAATTACTACCCCAACAG
CTTTGGTGCTCCGGAACAACAGCCTTCTGCCCTGGAGCACAGCATCCAAT
ATTCTGGAGAAGTGCGGAGATTCAACACTGCCAATGATGATAACGTTACT
CAGGTGCGGGCATTCTATGTGAACGTGCTGAATGAGGAACAGAGGAAAC
GTCTGTGTGAGAACATTGCCGGCCACCTGAAGGATGCACAAATTTTCATC
CAGAAGAAAGCGGTCAAGAACTTCACTGAGGTCCACCCTGACTACGGGA
GCCACATCCAGGCTCTTCTGGACAAGTACAATGCTGAGAAGCCTAAGAAT
GCGATTCACACCTTTGTGCAGTCCGGATCTCACTTGGCGGCAAGGGAGAA
GGCAAATCTG
encodes
(SEQ ID NO: 12)
APRAATMPRVRSCLLHSPRTHALADSRDPASDQMQHWKEQRAAQKADVLT
TGAGNPVGDKLNVITVGPRGPLLVQDVVFTDEMAHFDRERIPERVVHAKGA
GAFGYFEVTHDITKYSKAKVFEHIGKKTPIAVRFSTVAGESGSADTVRDPRGF
AVKFYTEDGNWDLVGNNTPIFFIRDPILFPSFIHSQKRNPQTHLKDPDMVWDF
WSLRPESLHQVSFLFSDRGIPDGHRHMNGYGSHTFKLVNANGEAVYCKFHY
KTDQGIKNLSVEDAARLSQEDPDYGIRDLFNAIATGKYPSWTFYIQVMTFNQ
AETFPFNPFDLTKVWPHKDYPLIPVGKLVLNRNPVNYFAEVEQIAFDPSNMPP
GIEASPDKMLQGRLFAYPDTHRHRLGPNYLHIPVNCPYRAPNYYPNSFGAPE
QQPSALEHSIQYSGEVRRFNTANDDNVTQVRAFYVNVLNEEQRKRLCENIAG
HLKDAQIFIQKKAVKNFTEVHPDYGSHIQALLDKYNAEKPKNAIHTFVQSGS
HLAAREKANLYPYDVPDYA;
or one having a Ig signal sequence
(SEQ ID NO: 13)
atgccacgcgtccgctcctgtcttctccacagtcccagaacacacgcactc
GCTGACAGCCGGGATCCCGCCAGCGACCAGATGCAGCAC
TGGAAGGAGCAGCGGGCCGCGCAGAAAGCTGATGTCCT
GACCACTGGAGCTGGTAACCCAGTAGGAGACAAACTTAA
TGTTACAGTAGGGCCCCGTGGGCCCCTTCTTGTTCAG
GATGTGGTTTTCACTGATGAAATGGCTCATTTTGACCGAG
AGAGAATTCCTGAGAGAGTTGTGCATGCTAAAGGAGCAG
GGGCCTTTGGCTACTTTGAGGTCACACATGACATTACCAA
ATACTCCAAGGCAAAGGTATTTGAGCATATTGGAAAGAAG
ACTCCCATCGCAGTTCGGTTCTCCACTGTTGCTGGAGAATC
GGGTTCAGCTGACACAGTTCGGGACCCTCGTGGGTTTGCA
GTGAAATTTTACACAGAAGATGGTAACTGGGATCTCGTTG
GAAATAACACCCCCATTTTCTTCATCAGGGATCCCATATTG
TTTCCATCTTTTATCCACAGCCAAAAGAGAAATCCTCAGAC
ACATCTGAAGGATCCGGACATGGTCTGGGACTTCTGGAGC
CTACGTCCTGAGTCTCTGCATCAGGTTTCTTTCTTGTTCAGT
GATCGGGGGATTCCAGATGGACATCGCCACATGAATGGA
TATGGATCACATACTTTCAAGCTGGTTAATGCAAATGGGG
AGGCAGTTTATTGCAAATTCCATTATAAGACTGACCAGGG
CATCAAAAACCTTTCTGTTGAAGATGCGGCGAGACTTTCC
CAGGAAGATCCTGACTATGGCATCCGGGATCTTTTTAACG
CCATTGCCACAGGAAAGTACCCGTCCTGGACTTTTTACATC
CAGGTCATGACATTTAATCAGGCAGAAACTTTTCCATTTA
ATCCATTCGATCTCACCAAGGTTTGGCCTCACAAGGACTA
CCCTCTCATCCCAGTTGGTAAACTGGTCTTAAACCGGAAT
CCAGTTAATTACTTTGCTGAGGTTGAACAGATAGCCTTCGA
CCCAAGCAACATGCCACCTGGCATTGAGGCCAGTCCTGAC
AAAATGCTTCAGGGCCGCCTTTTTGCCTATCCTGACACTCA
CCGCCATCGCCTGGGACCCAATTATCTTGATATACCTGTGA
ACTGTCCCTACCGTGCTCCAAATTACTACCCCAACAGCTTT
GGTGCTCCGGAACAACAGCCTTCTGCCCTGGAGCACAGC
ATCCAATATTCTGGAGAAGTGCGGAGATTCAACACTGCCA
ATGATGATAACGTTACTCAGGTGCGGGCATTCTATGTGA
ACGTGCTGAATGAGGAACAGAGGAAACGTCTGTGTGAG
AACATTGCCGGCCACCTGAAGGATGCACAAATTTTCATCC
AGAAGAAAGCGGTCAAGAACTTCACTGAGGTCCACCCTG
ACTACGGGAGCCACATCCAGGCTCTTCTGGACAAGTACA
ATGCTGAGAAGCCTAAGAATGCGATTCACACCTTTGTGC
AGTCCGGATCTCACTTGGCGGCAAGGGAGAAGGCAAATCTG
(SEQ ID NO: 11; DNA Sequence hSOD3hd⁻)
ATGCTGGCGCTACTGTGTTCCTGCCTGCTCCTGGCAGCCGGTGCCTCGGAC
GCCTGGACGGGCGAGGACTCGGCGGAGCCCAACTCTGACTCGGCGGAGT
GGATCCGAGACATGTACGCCAAGGTCACGGAGATCTGGCAGGAGGTCGC
CACGCTGGACGCCGCGCAGCACGCCGCCTGCCAGGTGCAGCCGTCGGCCA
CGCTGGACGCCGCGCAGCCCCGGGTGACCGGCGTCGTCCTCTTCCGGCAG
CTTGCGCCCCGCGCCAAGCTCGACGCCTTCTTCGCCCTGGAGGGCTTCCCG
ACCGAGCCGAACAGCTCCAGCCGCGCCATCCACGTGCACCAGTTCGGGGA
CCTGAGCCAGGGCTGCGAGTCCACCGGGCCCCACTACAACCCGCTGGCCG
TGCCGCACCCGCAGCACCCGGGCGACTTCGGCAACTTCGCGGTCCGCGAC
GGCAGCCTCTGGAGGTACCGCGCCGGCCTGGCCGCCTCGCTCGCGGGCCC
GCACTCCATCGTGGGCCGGGCCGTGGTCGTCCACGCTGGCGAGGACGACC
TGGGCCGCGGCGGCAACCAGGCCAGCGTGGAGAACGGGAACGCGGGCCG
GCGGCTGGCCTGCTGCGTGGTGGGC
encodes
(SEQ ID NO: 10)
MLALLCSCLLLAAGASDAWTGEDSAEPNSDSAEWIRDMYAKVTEIWQEVAT
LDAAQHAACQVQPSATLDAAQPRVTGVVLFRQLAPRAKLDAFFALEGFPTEP
NSSSRAIHVHQFGDLSQGCESTGPHYNPLAVPHPQHPGDFGNFAVRDGSLWR
YRAGLAASLAGPHSIVGRAVVVHAGEDDLGRGGNQASVENGNAGRRLACC
VVG

Sequences within the scope of the invention include those with at least 80%, 85%, 88%, 90%, 92%, 94%, 95%, 96%, 97%, 98%, 99% or more amino acid sequence identity to one of SEQ ID Nos. 1-8, 10 or 12. Catalase sequences within the scope of the invention have about 2%, 5%, 10%, 12%, 15% or up to 20% fewer residues than a full length catalase sequence. Superoxide dismutase sequences within the scope of the invention have about 2%, 5%, 10%, 12%, 15% or up to 20% fewer residues than a full length superoxide dismutase sequence.

### Gene Delivery Vectors

Gene delivery vectors include, for example, viral vectors, liposomes and other lipid-containing complexes, such as lipoplexes (DNA and cationic lipids), polyplexes, e.g., DNA complexed with cationic polymers such as polyethylene glycol, nanoparticles, e.g., magnetic inorganic nanoparticles that bind or are functionalized to bind DNA such as Fe₃O₄ or MnO₂ nanoparticles, microparticles, e.g., formed of polylactide polygalactide reagents, nanotubes, e.g., silica nanotubes, and other macromolecular complexes capable of mediating delivery of a gene to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. A large variety of such vectors are known in the art and are generally available.

Gene delivery vectors within the scope of the invention include, but are not limited to, isolated nucleic acid. e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary viral gene delivery vectors are described below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch. In one embodiment, a permeation enhancer is not employed to enhance indirect delivery to the CNS.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing neural specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans. Moreover, they appear promising for sustained cardiac gene transfer (Hoshijima et al., Nat. Med., 8:864 (2002); Lynch et al., Circ. Res., 80:197 (1997)).

AAV vectors include but are not limited to AAV1, AAV2, AAV5, AAV7, AAV8, AAV9 or AAVrh.10.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. For example, expression of secreted angiogenesis factors after muscle injection of plasmid DNA, despite relatively low levels of focal transgene expression, has demonstrated significant biologic effects in animal models and appears promising clinically (Isner, Nature, 415:234 (2002)). Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

### Pharmaceutical Compositions

The invention provides a composition comprising, consisting essentially of, or consisting of the above-described gene transfer vector(s) and a pharmaceutically acceptable (e.g., physiologically acceptable) carrier. When the composition consists essentially of the inventive gene transfer vector and a pharmaceutically acceptable carrier, additional components can be included that do not materially affect the composition (e.g., adjuvants, buffers, stabilizers, anti-inflammatory agents, solubilizers, preservatives, etc.). When the composition consists of the inventive gene transfer vector and the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition optionally can be sterile with the exception of the gene transfer vector described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the inventive gene transfer vector is administered in a composition formulated to protect the gene transfer vector from damage prior to administration. For example, the composition can be formulated to reduce loss of the gene transfer vector on devices used to prepare, store, or administer the gene transfer vector, such as glassware, syringes, or needles. The composition can be formulated to decrease the light sensitivity and/or temperature sensitivity of the gene transfer vector. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the gene transfer vector, facilitate administration, and increase the efficiency of the inventive method. Formulations for gene transfer vector-containing compositions are further described in, for example, Wright et al., Curr. Opin. Drug Discov. Devel., 6(2): 174-178 (2003) and Wright et al., Molecular Therapy, 12: 171-178 (2005))

The composition also can be formulated to enhance transduction efficiency. In addition, one of ordinary skill in the art will appreciate that the inventive gene transfer vector can be present in a composition with other therapeutic or biologically-active agents. For example, factors that control inflammation, such as ibuprofen or steroids, can be part of the composition to reduce swelling and inflammation associated with in vivo administration of the gene transfer vector. Immune system stimulators or adjuvants. e.g., interleukins, lipopolysaccharide, and double-stranded RNA. Antibiotics. i.e., microbicides and fungicides, can be present to treat existing infection and/or reduce the risk of future infection, such as infection associated with gene transfer procedures.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see. e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the inventive gene transfer vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the gene transfer vector in the composition administered to the mammal will depend on a number of factors, including the size (mass) of the mammal, the extent of any side-effects, the particular route of administration, and the like. In one embodiment, the inventive method comprises administering a “therapeutically effective amount” of the composition comprising the inventive gene transfer vector described herein. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the gene transfer vector to elicit a desired response in the individual. The dose of gene transfer vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene transfer vector dose range to treat a patient having a particular disease or disorder, based on these and other factors that are well known in the art. The therapeutically effective amount may be between 1×10¹⁰ genome copies to 1×10¹³ genome copies.

In one embodiment, the composition is administered once to the mammal. It is believed that a single administration of the composition may result in persistent expression in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of gene transfer vector comprising a nucleic acid sequence as described above.

### Routes of Administration, Dosages and Dosage Forms

Administration of the gene delivery vectors in accordance with the present invention may be continuous or intermittent, depending, for example, upon the recipient's physiological condition, and other factors known to skilled practitioners. The administration of the gene delivery vector(s) may be essentially continuous over a preselected period of time or may be in a series of spaced doses. Both local administration, e.g., intracranial, intranasal or intrathecal, and systemic administration, e.g., using viruses that cross the blood-brain barrier, are contemplated. Any route of administration may be employed, e.g., intravenous, intranasal or intrabronchial, direct administration to the lung and intrapleural. In one embodiment, compositions may be delivered to the pleura.

One or more suitable unit dosage forms comprising the gene delivery vector(s), which may optionally be formulated for sustained release, can be administered by a variety of routes including intracranial, intrathecal, or intranasal, or other means to deliver to the CNS, or oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

The amount of gene delivery vector(s) administered to achieve a particular outcome will vary depending on various factors including, but not limited to, the genes and promoters chosen, the condition, patient specific parameters, e.g., height, weight and age, and whether prevention or treatment, is to be achieved.

Vectors of the invention may conveniently be provided in the form of formulations suitable for administration, e.g., into the brain. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Vectors of the present invention may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions of the invention can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors can be provided in a dosage form containing an amount of a vector effective in one or multiple doses. For viral vectors, the effective dose may be in the range of at least about 10⁷ viral particles, e.g., about 10⁹ viral particles, or about 10¹¹ viral particles. The number of viral particles added may be up to 10¹⁴. For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results. The amount administered will vary depending on various factors including, but not limited to, the nucleic acid or vector chosen for administration, the disease, the weight, the physical condition, the health, and/or the age of the mammal. Such factors can be readily determined by the clinician employing animal models or other test systems that are available in the art. As noted, the exact dose to be administered is determined by the attending clinician, but may be in 1 mL phosphate buffered saline. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered.

For example, when a viral expression vector is employed, about 10 to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

In one embodiment, administration may be by intracranial, intrahepatic, intratracheal or intrabronchial injection or infusion using an appropriate catheter or needle. A variety of catheters may be used to achieve delivery, as is known in the art. For example, a variety of general purpose catheters, as well as modified catheters, suitable for use in the present invention are available from commercial suppliers. Also, where delivery is achieved by injection directly into a specific region of the brain or lung, a number of approaches can be used to introduce a catheter into that region, as is known in the art.

By way of illustration, liposomes and other lipid-containing gene delivery complexes can be used to deliver one or more transgenes. The principles of the preparation and use of such complexes for gene delivery have been described in the art (see, e.g., Ledley, (1995); Miller et al., (1995); Chonn et al., (1995); Schofield et al., (1995); Brigham et al., (1993)).

Pharmaceutical formulations containing the gene delivery vectors can be prepared by procedures known in the art using well known and readily available ingredients. For example, the agent can be formulated with common excipients, diluents, or carriers, and formed into tablets, capsules, suspensions, powders, and the like. The vectors of the invention can also be formulated as elixirs or solutions appropriate for parenteral administration, for instance, by intramuscular, subcutaneous or intravenous routes.

The pharmaceutical formulations of the vectors can also take the form of an aqueous or anhydrous solution, e.g., a lyophilized formulation, or dispersion, or alternatively the form of an emulsion or suspension.

In one embodiment, the vectors may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the vector is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the vector may be administered via nose drops, a liquid spray, such as via a plastic bottle atomizer or metered-dose inhaler. Typical of atomizers are the Mistometer (Wintrop) and the Medihaler (Riker).

The local delivery of the vectors can also be by a variety of techniques which administer the vector at or near the site of disease. e.g., using a catheter or needle Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans. Native Americans. Semites. and Pacific Islanders. The methods may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

The invention will be described by the following non-limiting examples.

### EXAMPLES

The present description is further illustrated by the following examples, which should not be construed as limiting in any way. The contents of all cited references (including literature references, issued patents, published patent applications as cited throughout this application) are hereby expressly incorporated by reference.

The animal experiments are designed to guarantee unbiased experimental design. Experimental animals will be randomly assigned to groups, and the investigators will be blinded when evaluating animal behavior. Males and females will be used to address possible gender differences in transduction, disease manifestation, or therapy response. The numbers of animals in each cohort have been chosen to yield statistically significant data.

### Overview

The focus of this disclosure is the development of therapy to protect the lung with an extracellular anti-oxidant defense to treat disorders including chronic obstructive pulmonary disease (COPD), a chronic disorder in which inhaled oxidants from tobacco smoke, pollution and generated within the lung by activated inflammatory cells play a major role in perpetuating injury to the lung epithelium and endothelium fundamental to the pathogenesis of the disease. The strategy is to use in vivo gene therapy technology to provide a persistent extracellular anti-oxidant enzyme shield to the lung that will inactivate superoxide (O₂⁺) and H₂O₂, major extracellular oxidant stresses to the lung. The strategy includes the use of genes for catalase and superoxide dismutase 3 (SOD3) that have been genetically modified to secrete functional monomeric antioxidant enzymes that can diffuse in the extracellular milieu, providing the lung with an effective extracellular anti-oxidant shield. Catalase is a tetrameric intracellular enzyme that is too large (232 kDa) to diffuse if designed to be secreted. To use catalase as an effective extracellular anti-oxidant, the catalase gene was modified to prevent the wrapping loop domain to mediate tetramer formation. With the addition of a secretory signal, the human catalase monomer (hCatWL⁻) is secreted, capable of functioning to catalyze extracellular H₂O₂ to H₂O. Superoxide dismutase 3 (SOD3) is secreted, but it is a large tetramer (130 kDa), and has a heparin-binding domain that attaches it to cell surfaces. To modify SOD3 into a more effective lung extracellular antioxidant, a loop critical for tetramer formation was modified and the heparin-binding domain removed (hSOD3hd⁻), resulting in an effective monomer antioxidant enzyme (30 kDa) that will not bind to cell surfaces. Adeno-associated (AAV) gene transfer vectors are used as exemplary gene transfer vectors to genetically modify the liver to express and secrete the modified catalase and/or SOD3 monomers. Four AAVrh.10 candidates will be evaluated: AAVrh.10hCatWL⁻ (expressing the catalase monomer); AAVrh.10hSOD3hd⁻ (SOD3 monomer); AAVrh.10hCatWL⁻/hSOD3hd⁻ (both monomers); and AAVrh.10hSODhd⁻/hCatWL⁻ (same but with SOD3hd⁻ in the 5′ position). In one embodiment an AAVrh.10 vector generates a persistent extracellular antioxidant shield of the lung endothelial and epithelial surface after administration.

Aim 1. To compare in vitro the levels of expression of secreted, functional modified catalase and/or SOD3, mediated by the expression cassette of the 4 AAVrh.10 antioxidant vectors (hCatWL⁻, hSOD3hd⁻, hCatWL⁻/hSOD3hd⁻ and hSOD3hd⁻/hCatWL⁻).

Aim 2. To quantify in vivo the ability of the 4 AAVrh.10 antioxidant vectors to express secreted, functional modified catalase and/or SOD3 capable of protecting lung endothelium and epithelium from O₂⁺ and/or H₂O₂ stress.

### Example 1

The focus of gene therapy disclosed herein is, in one embodiment, to protect the lung with an extracellular anti-oxidant defense to treat chronic obstructive pulmonary disease (COPD), a chronic disorder in which inhaled oxidants from tobacco smoke, pollution and generated within the lung by activated inflammatory cells play a major role in initiating and perpetuating injury to the lung epithelium and endothelium fundamental to pathogenesis of the disease (Lin & Thomas, 2010; McGuinness et al., 2017; Hubbard & Crystal, 1986; MacNee, 2000; Shapiro & Ingenito, 2005; Yoshida et al., 2007; Elmasry et al., 2015). Much of the oxidant stress on the lung is extracellular, overwhelming the intracellular anti-oxidant defenses of epithelial and endothelial cells, resulting in cell damage, dysfunction and eventual death (Shaykhiev et al., 2014; Gao et al., 2015; Polverino et al., 2018). The current strategy is to use in vivo gene therapy technology to provide a persistent extracellular anti-oxidant enzyme shield to the lung that will inactivate superoxide (02) and H₂O₂, two major components of extracellular oxidant stress to the lung.

The coding sequences for catalase and superoxide dismutase 3 (SOD3) have been genetically modified to be secreted in functional monomeric forms that can diffuse in the extracellular milieu, providing the lung with an effective extracellular anti-oxidant shield. Catalase is a large (232 kDa) tetrameric enzyme that combined with heme groups and NADPH is a highly effective intracellular enzyme, but too large to effectively diffuse in the extracellular milieu even if designed to be secreted (Reynolds et al., 1977; Rennard et al., 1986; Goyal & Basak, 2010; Sepasi et al., 2018; Bell et al., 1981). To use catalase as an effective extracellular anti-oxidant, the catalase gene was modified to prevent the wrapping loop domain to mediate tetramer formation (Goyal & Basak, 2010; Ko et al., 2000; Safo et al., 2001). With the addition of a secretory signal, the human catalase monomer (hCatWL⁻) is secreted, capable of functioning to catalyze extracellular H₂O₂ to H₂O.

There are 3 superoxide dismutase genes. SOD1, 2 and 3 (Fukai & Ushio-Fukai, 2011; Perry et al., 2010). SOD1 functions in the cytoplasm and SOD2 in the mitochondria. SOD3 is secreted, but is a large tetramer (130 kDa), and has a heparin-binding domain that attaches it to cell surfaces (Fukai & Ushio-Fukai, 2011; Antonyuk et al., 2009; Griess et al., 2017). To modify SOD3 into a more effective lung extracellular antioxidant, a loop critical for tetramer formation was modified and the heparin-binding domain removed (hSOD3hd), resulting in a functional, secreted monomer antioxidant that will not bind to cell surfaces. In one embodiment, adeno-associated (AAV) gene transfer vectors are used to genetically modify the liver to express and secrete the modified catalase and/or SOD3 monomers, each with a molecular mass capable of diffusing across the lung (Reynolds et al., 1977; Bell et al., 1981), the result is extracellular anti-oxidant protection for the entire lung. In one embodiment, the gene therapy strategy use sAAVrh.10, a nonhuman primate adeno-associated virus (AAV) gene transfer vector that, when administered intravenously effectively transduces hepatocytes to express the antioxidant gene(s). Four AAVrh.10 candidates are evaluated: AAVrh.10hCatWL⁻ (expressing only the catalase monomer); AAVrh.10hSOD3hd⁻ (SOD3 monomer); AAVrh.10hCatWL⁻/hSOD3hd⁻ (both antioxidant monomers); and AAVrh.10hSODhd⁻/hCatWL⁻ (the same but with SOD3hd⁻ in the 5′ position).

Oxidants are molecules that readily accept electrons from other molecules causing dysfunction and eventual cell/organ or damage (Davies et al., 2001; Devasagayam et al., 2002; O'Reilly et al., 2001; Janssen et al., 1993). Under normal circumstances, antioxidants protect against oxidative stress (Pham-Huy et al., 2008; Irshad et al., 2002). When the oxidant burden outweighs the antioxidant defenses, the resulting oxidant stress plays a central role in the pathogenesis of organ dysfunction (Casas et al., 2015; Pham-Huy et al., 2008; Irshad et al., 2002). While the lung is highly vulnerable to inhaled extracellular oxidants (tobacco smoke, pollutants, xenobiotics, hyperoxia) and endogenous oxidants (activated inflammatory cells), the lung antioxidant defenses are primarily intracellular (Rhaman et al., 2006; Sies et al., 2017).

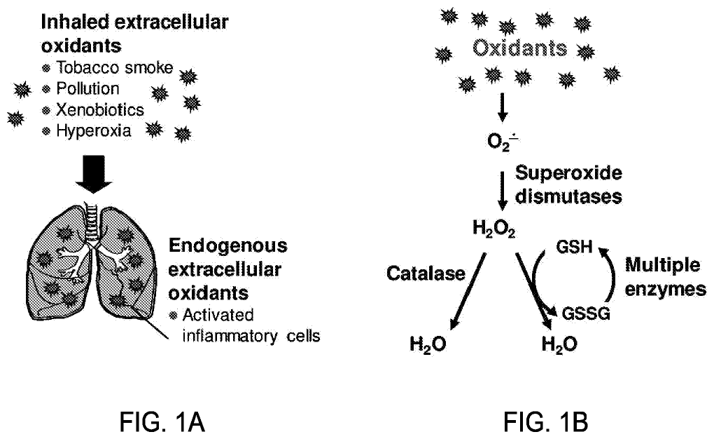

In COPD, the lung epithelium and endothelium are under additional oxidant stress from activated inflammatory cells (alveolar macrophages and neutrophils) that generate extracellular oxidants that overwhelm intracellular oxidant defenses (FIG. 1A). The major lung antioxidant defense enzymes are superoxide dismutase (SOD; catalyzes O₂⁺ to H₂O₂; 3 forms: SOD1 cytoplasmic, SOD2 mitochondrial, SOD3 extracellular), catalase (catalyzes H₂O₂ to H₂O, cytoplasmic), and the glutathione system converts H₂O₂ to water (GSH is both cytoplasmic and extracellular; multiple cytoplasmic enzymes are required to keep GSH reduced; FIG. 1B) (Rahman et al., 2006; Sies et al., 2017). Although lung cells have oxidant sensors such as NRF2 and NF-kB/IkB, human lung cells are not capable of up-regulating the major antioxidant enzymes catalase and SOD. This was dramatically demonstrated in a study carried out by the Crystal laboratory, in which normal human volunteers were bronchoscoped to sample airway epithelium to quantify baseline catalase and SOD mRNA levels, and then after exposure to 100% 02 for 12-18 hours (sufficient to induce tracheobronchitis), the epithelium was resampled (Erzurum et al., 1985). Strikingly, neither catalase nor SOD mRNA levels were up-regulated. i.e., the human airway epithelium has limited mechanisms to up-regulate its antioxidant shield despite intense extracellular oxidant stress. One solution is to provide an effective extracellular “antioxidant Teflon coat” to protect the lung from the stress of extracellular oxidants. An adeno-associated virus (AAV) gene transfer vector is used to express a secreted form of catalase alone, SOD3 alone or catalase+SOD3 to generate an effective anti-oxidant shield. The challenge is that catalase is a large intracellular tetramer and SOD3 is a large tetramer that binds to cell surfaces; in their natural form, neither can provide effective diffuse extracellular antioxidant defense. The solution is to modify the coding sequences of catalase and SOD3 to generate functional monomers that can be used effectively as gene therapy based antioxidants capable of diffusing through the lung, protecting the oxidant-vulnerable endothelium and epithelium (Reynolds et al., 1977; Bell et al., 1981). By administering, in one embodiment, the AAV vector coding for the modified catalase and/or SOD3 monomers intravenously, liver hepatocytes will secrete the functional-antioxidant monomers into blood, enabling antioxidant protection of the pulmonary endothelium (from the blood side) and of sufficiently low molecular weights (50-60 kDa) to diffuse across the endothelium and epithelial tight junctions to provide an effective antioxidant shield of the interstitial tissues and epithelium (for proteins of molecular weight 50-60 kDa, the human lung epithelial lining fluid levels are 10% that of the blood

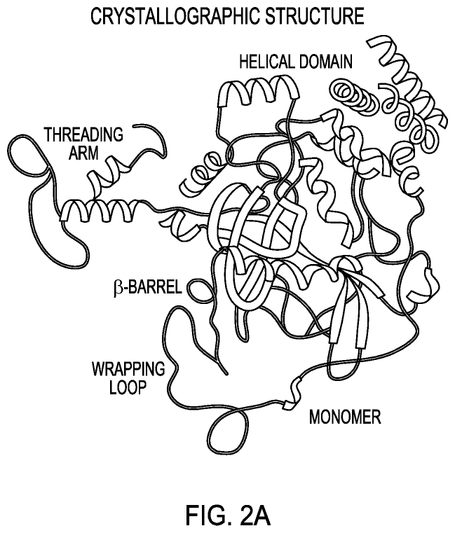

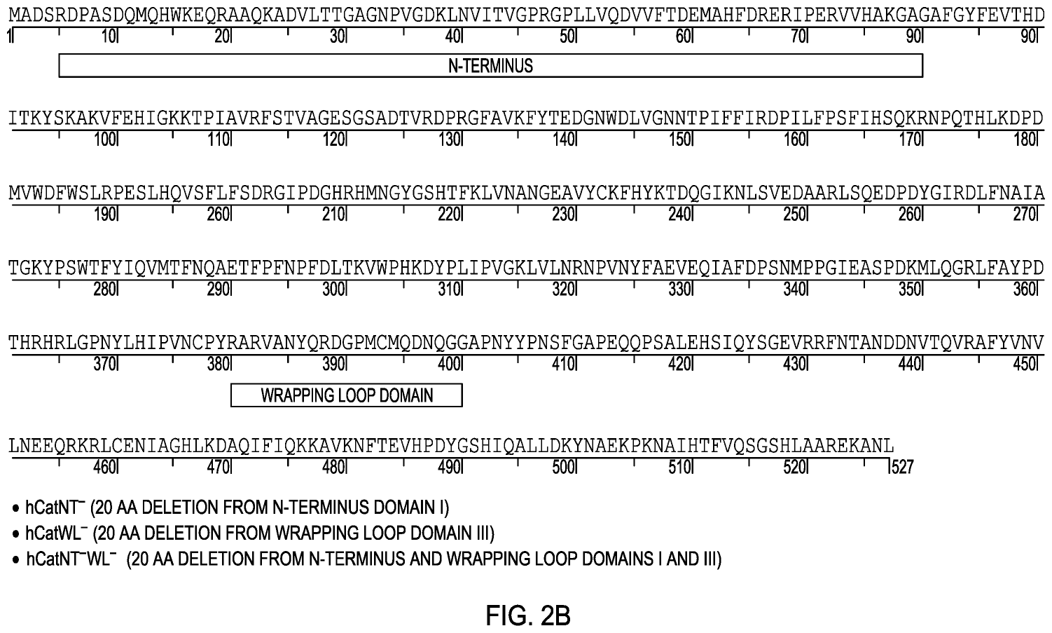

One catalase molecule can convert millions of H₂O₂ molecules to H₂O each second (Goyal & Basak, 2010; Chance, 1947). Catalase is an intracellular enzyme comprised of 4 monomers (each 501 amino acids)+4 iron-containing 4 heme groups+4 NAPDH molecules. Catalase is expressed in all organs. The challenge for a gene therapy strategy to boost lung extracellular antioxidant protection is that catalase is too large (232 kDa) to secrete and diffuse to provide an effective extracellular antioxidant shield. The LEX solution is to genetically modify the catalase gene sequence such that it cannot form tetramers, and can be secreted as a monomer that will function as an effective extracellular antioxidant. Each catalase monomer has amino-terminal residues important for interlocking arm exchange that binds the monomeric units together, and a wrapping loop domain—the 4 monomers wrap around each other to form a tetramer, with salt bridges and ionic interactions holding the 4 monomers together. The candidate genetic modifications included: (1) all constructs had a N-terminal secretion sequence (from human IgG1); (2) the catalase sequence was modified in the N-terminus region to delete a domain that stabilizes the tetramer structure; and (3) the wrapping loop domain that forms the binding interface between monomers was deleted (FIG. 2).

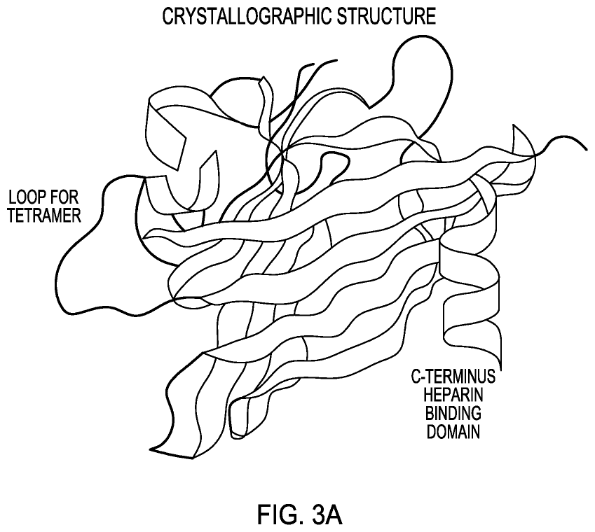

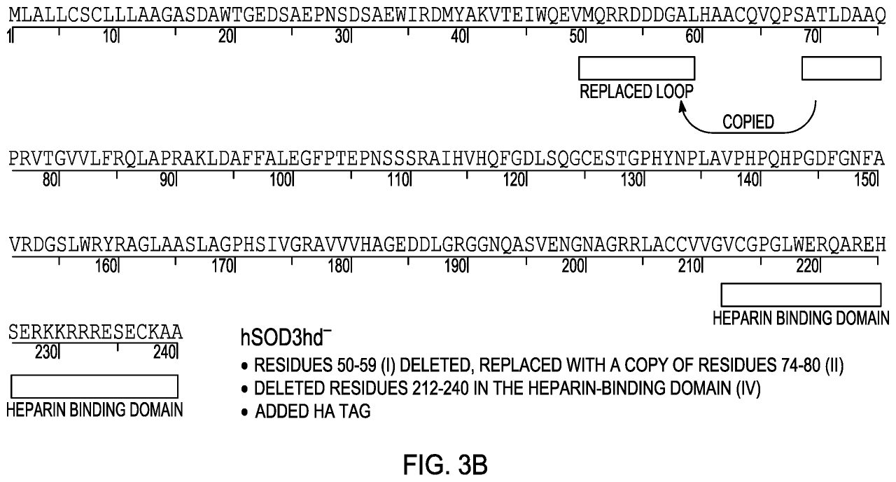

SOD converts O₂⁺ into H₂O₂. SOD3 (active site copper+zinc) is a secreted homotetramer (about 130 kDa) with an amino-terminal signal peptide and a C-terminal to heparin-binding domain comprised of a cluster of positively charged residues. Although secreted, the heparin-binding domain anchors SOD3 to the cell surface and to matrix heparin sulfate proteoglycan and collagen (Sandstom, 1993; Olsen et al., 2004) (a small fraction is cleaved near the N-terminus to generate circulating tetramers). To maximize SOD3 effectiveness as a gene therapeutic in the lung, a modification was made to replace a loop that engages monomers to form the tetramer, and a segment of the heparin-binding domain was deleted to allow the SOD3 monomer to freely diffuse in the tissue. Since SOD3 has a signal peptide, it was left intact (FIG. 3).

COPD is the 3^{rd} most common cause of death in the US. Other than oxygen, there are no drugs that decrease COPD-associated mortality (Benton et al., 2018; Woodruff et al., 2015). The drugs used in COPD (bronchodilators, corticosteroids) help symptoms and long-term antibiotics reduce the frequency of exacerbations (Benton et al., 2018; Woodruff et al., 2015). There is extensive data supporting the concept that the stress of extracellular oxidants plays a major role in the pathogenesis of COPD (Shaykiev et al., 2014; Gao et al., 2015; Polverino et al., 2010; Rahman, 2015). There have been several clinical studies to evaluate antioxidants for COPD therapy (reviewed in Rahman, 2008; Rahman, 2012). None of these trials have been successful. The LEX strategy is a new approach, using gene therapy technology to augment the levels of effective antioxidant enzymes in the extracellular milieu, to protect both the epithelial and endothelial cells from oxidant stress. If successful, in the context of the importance of oxidants in the pathogenesis of COPD, a gene therapy-based establishment of an extracellular antioxidant screen should be highly significant as a therapeutic for this common, fatal disorder (Foronjy et al., 2008; Rahman et al., 2006).

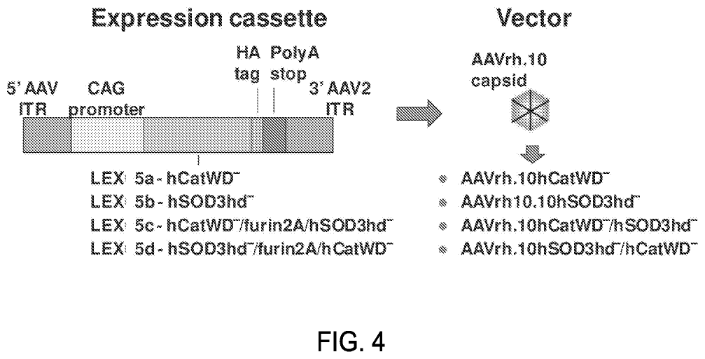

FIG. 4 shows an exemplary gene therapy-based, extracellular, diffusable enzymatic antioxidant to treat COPD. Based on studies to date, LEX 5 will be comprised of an AAV serotype rh.10 capsid, with 1 of 4 candidate expression cassettes (FIG. 4). The 4 candidate gene transfer vectors are identical except for the cDNA coding for the antioxidant enzyme. Each comprises an expression cassette with (5′ to 3′): (1) AAV inverted terminal repeat (ITR) from AAV serotype 2; (2) the CAG-cytomegalovirus enhancer/promoter, a splice donor, intron sequence from chicken β-actin, right hand intron and splice acceptor from rabbit β-globin; CAG is a highly active constitutive promoter widely used in gene therapy applications (Miyazaki et al., 1989; Niwa et al., 1991); (3) coding sequence of the catalase monomer (LEX 5a, hCatWD⁻), the SOD3 monomer (LEX 5b, hSOD3hd), the combined coding sequence (5′ to 3′) of hCatWD⁻+hSOD3hd⁻, separated by a furin 2A site (LEX 5c), and the combined coding (5′ to 3′) of hSOD3hd⁻+hCatWD⁻, separated by a furin 2A site (LEX 5d, identical to LEX 5c except the order of the catalase and SOD3 sequences are reversed); (4) hemagglutinin tag (to facilitate detection of the protein); (5) polyA/stop signal: and (6) 3′ AAV2ITR. The expression cassettes will all be packaged in the AAVrh.10 serotype capsid, a nonhuman primate AAV capsid which is excellent at transducing the liver to express secreted proteins The 4 candidate expression cassettes will be assessed in vitro for function and the 4 candidate AAV vectors will be compared head-to-head in mice, using equivalent intravenous doses of each vector, assessing the liver and lung for vector DNA and human catalase and/or SOD3 mRNA, and catalase monomer protein amount and activity in plasma (for pulmonary endothelial protection) and lung epithelial lining fluid (for pulmonary epithelial protection). Using quantitative criteria detailed in 3c, one of the vectors will be chosen from the 4 candidate vectors.

Globally, COPD affects 329 million (4.8% of the world population (Vos et al., 2012)) and causes over 3 million deaths worldwide each year. In the US, COPD is the 3^{rd} leading cause of death, causing an estimated 150,000 deaths per year. The vector may require only a single intravenous infusion for life-long therapy for this chronic, fatal disorder, rather than using gene therapy to express the native genes coding for antioxidant enzymes that primarily boost intracellular antioxidant (catalase) or remain attached to cell membranes (SOD3), the present strategy is to use gene therapy to generate an effective extracellular enzymatic antioxidant “shield.” providing extracellular antioxidant protection to all lung cells, including the highly vulnerable endothelium and epithelium.

The solution to generate an effective lung extracellular defense is to modify the native human catalase and SOD3 coding sequences so they can generate functional monomers of molecular weight (50-60 kDa) that, when expressed by AAV-mediated gene transfer to the liver, the monomers enhance blood antioxidant defenses (protecting the pulmonary endothelium) and diffuse across the lung and enhance lung epithelial lining fluid antioxidant defenses (protecting the epithelium). In summary, the innovations include: (1) molecular engineering of the human catalase and SOD3 genes to direct these highly effective antioxidants to create an extracellular antioxidant shield for lung endothelium and epithelium, preventing extracellular oxidant stress to damage lung cells; and (2) using in one embodiment a combination of the genetically modified catalase and SOD3 genes in one gene transfer construct takes advantage of the ability of these antioxidants to effectively remove both O₂⁺ and H₂O₂, thus providing an effective extracellular antioxidant shield against a broad spectrum of inhaled oxidants as well as extracellular oxidants generated by activated inflammatory cells. Separate catalase and SOD vectors may be administered together.

Based on the data, 4 candidate vectors have been identified, all based on AAV nonhuman serotype rh.10 (FIG. 4): (1) LEX 5a—AAVrh.10hCatWD⁻ (secreted catalase monomer); (2) LEX 5b—AAVrh.10hSOD3hd⁻ (secreted SOD3 monomer with the deleted heparin-binding domain); (3) LEX 5c—AAVrh.10hCatWD⁻/SOD3hd⁻ (single vector expressing both the secreted catalase monomer and the secreted SOD3 monomer); and (4) LEX 5d—AAVrh.10hSOD3hd⁻/hCatWD⁻ (identical to LEX 5c, but with the SOD3 construct proceeding the catalase construct). The experimental approach to identify LEX 5 uses in vitro and in vivo assays.

Modifications to the human catalase coding sequence led to the identification of the expression cassette for LEX 5a, creating AAVrh.10hCatWD⁻ (LEX 5a), and it was demonstrated that when LEX 5a is administered intravenously to mice, the result is functional human catalase activity in serum. Similarly, modifications to the human SOD3 coding sequence led to the identification of the expression cassette for LEX 5b to mice resulted in functional SOD activity in serum. The AAVrh.10hCatWD⁻/hSOD3hd⁻ and AAVrh.10hSOD3hd⁻/hCatWD⁻ vectors have been generated and are being tested in vitro and in vivo.

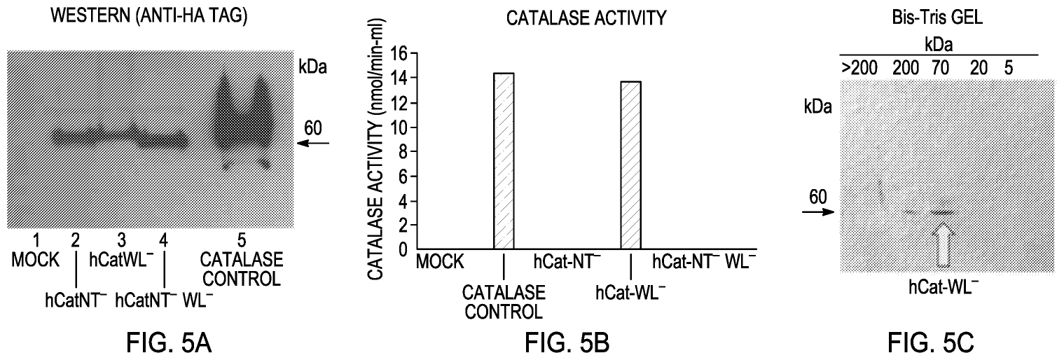

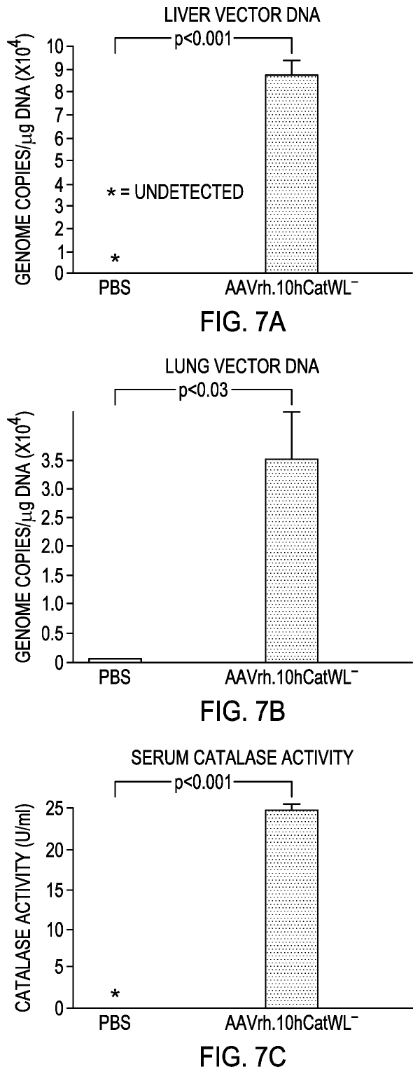

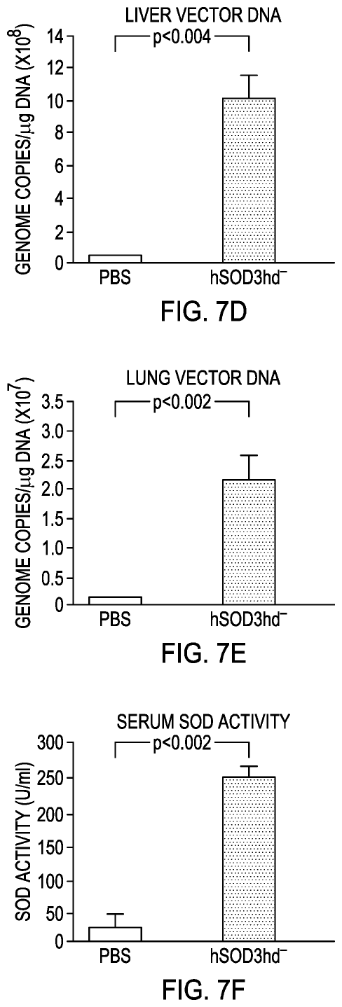

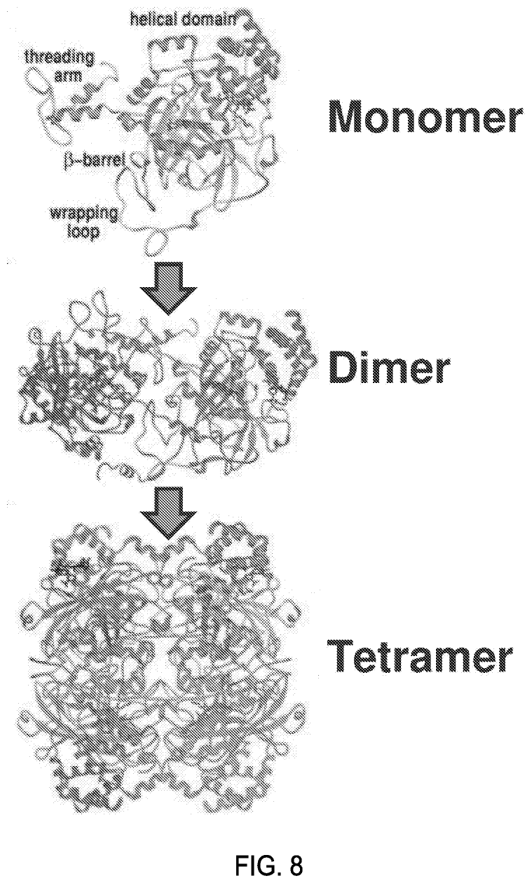

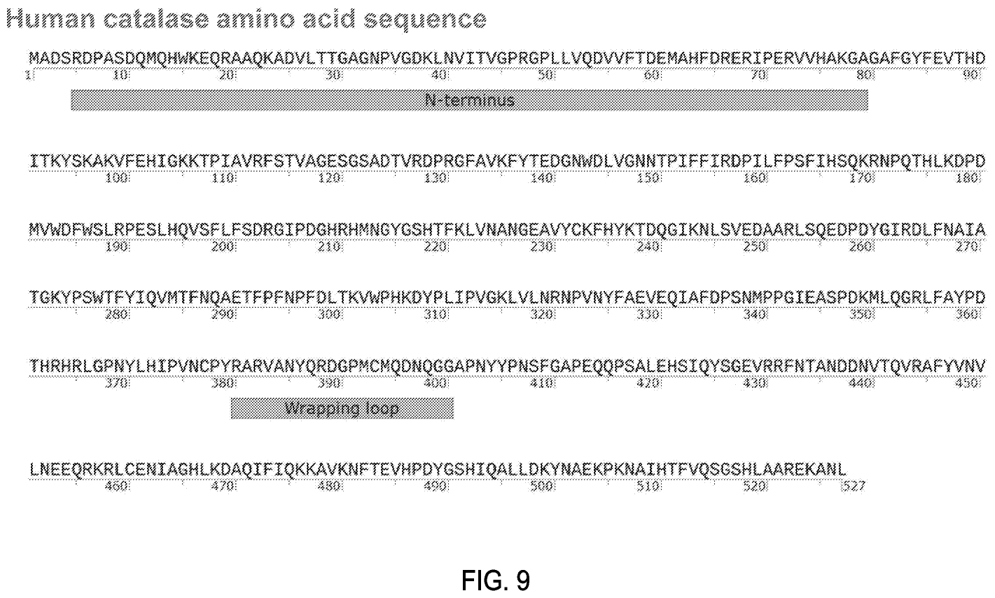

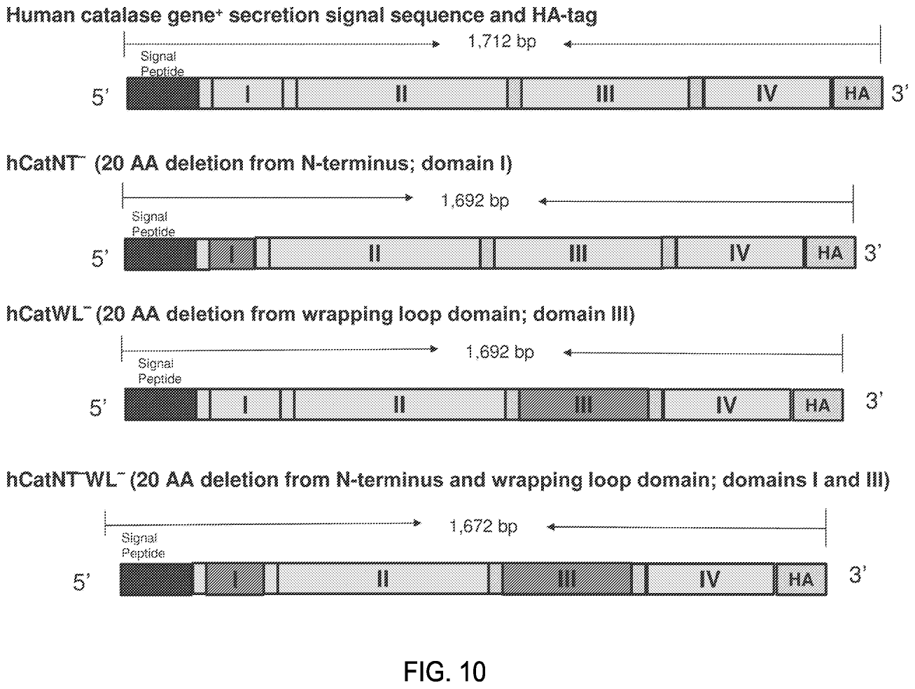

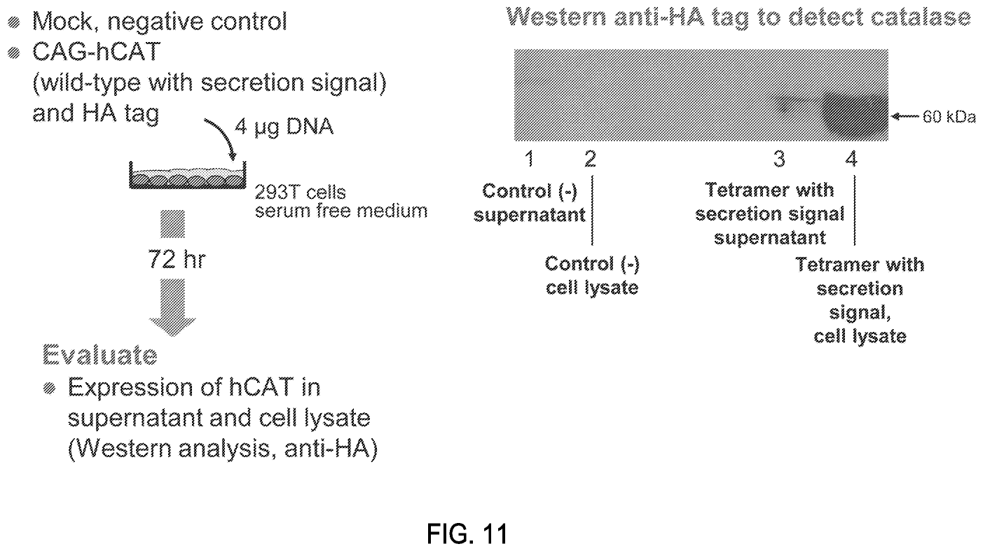

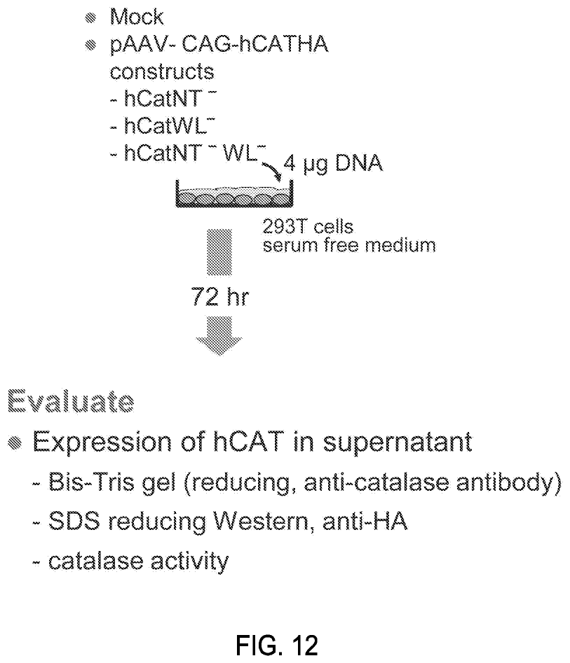

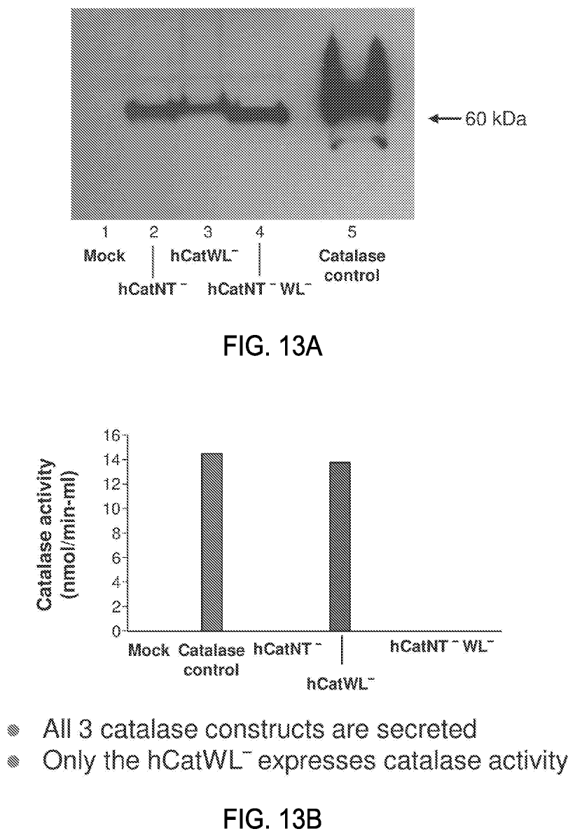

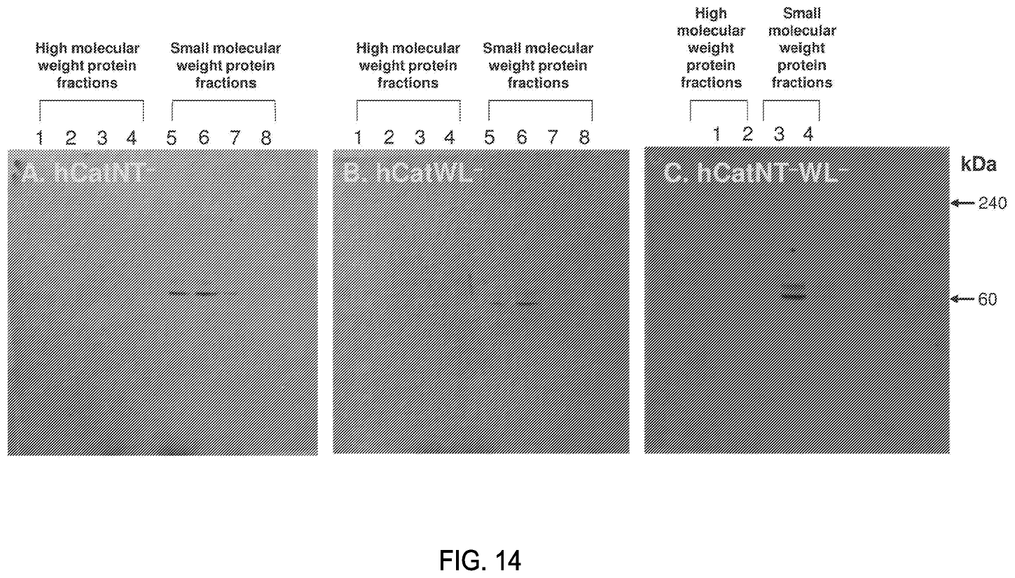

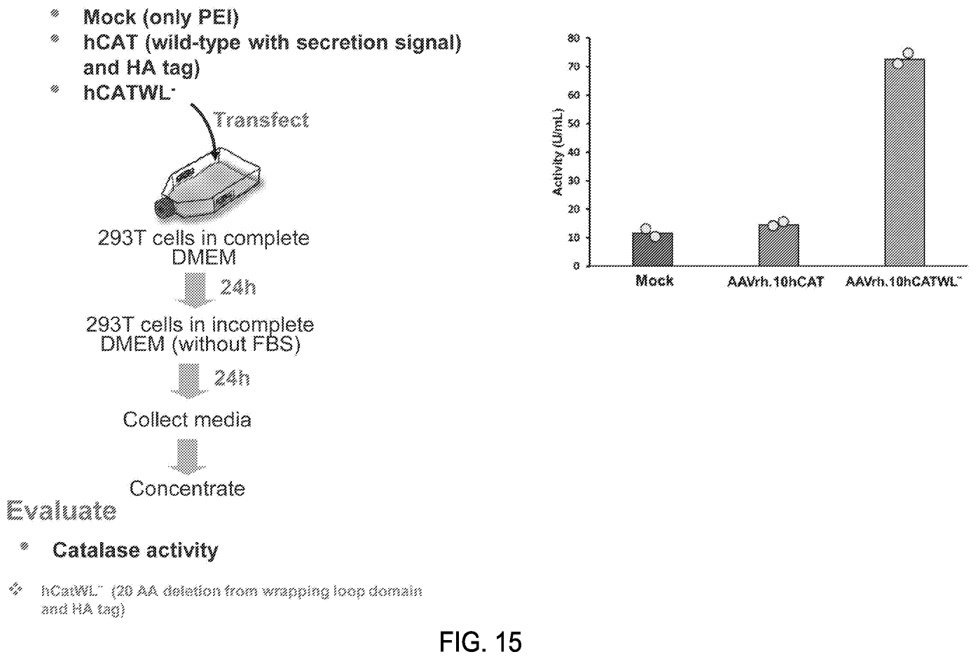

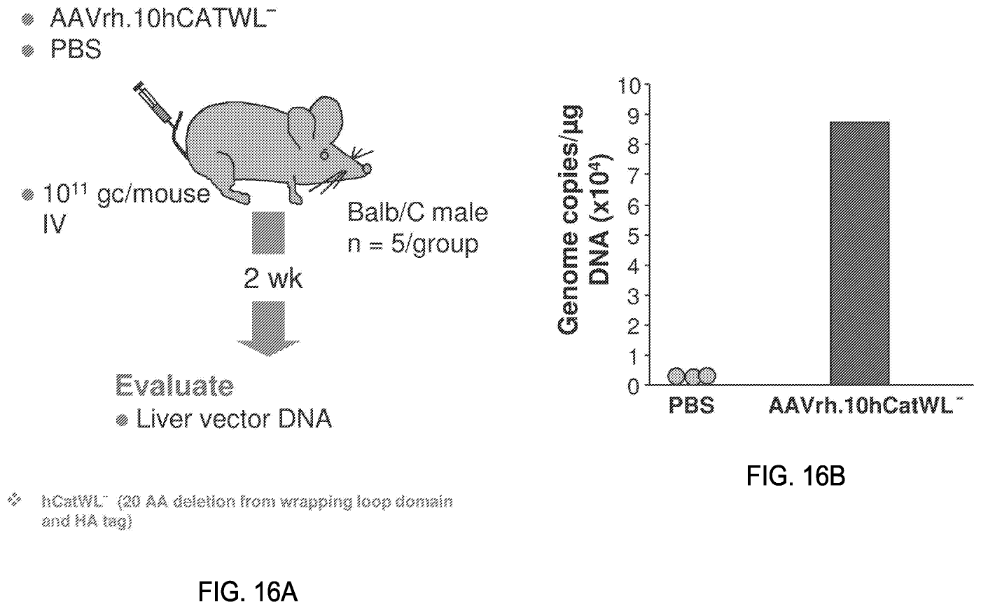

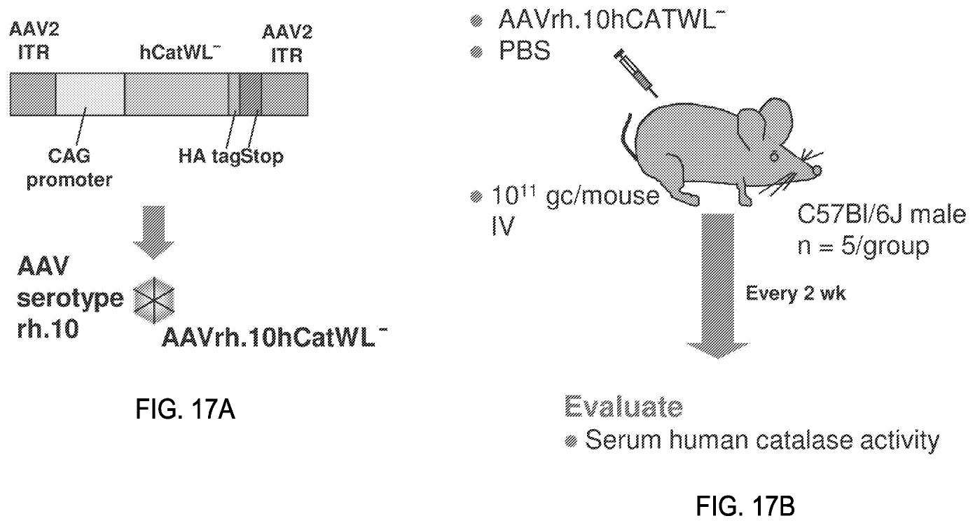

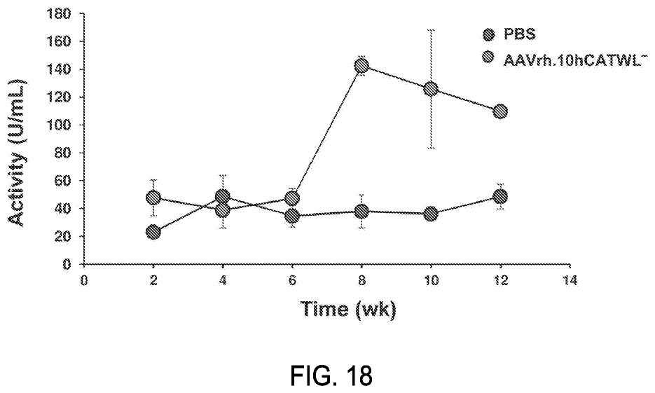

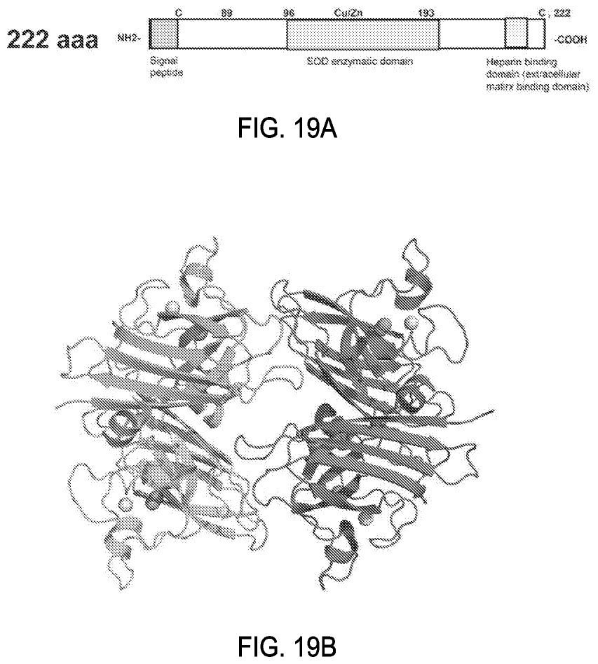

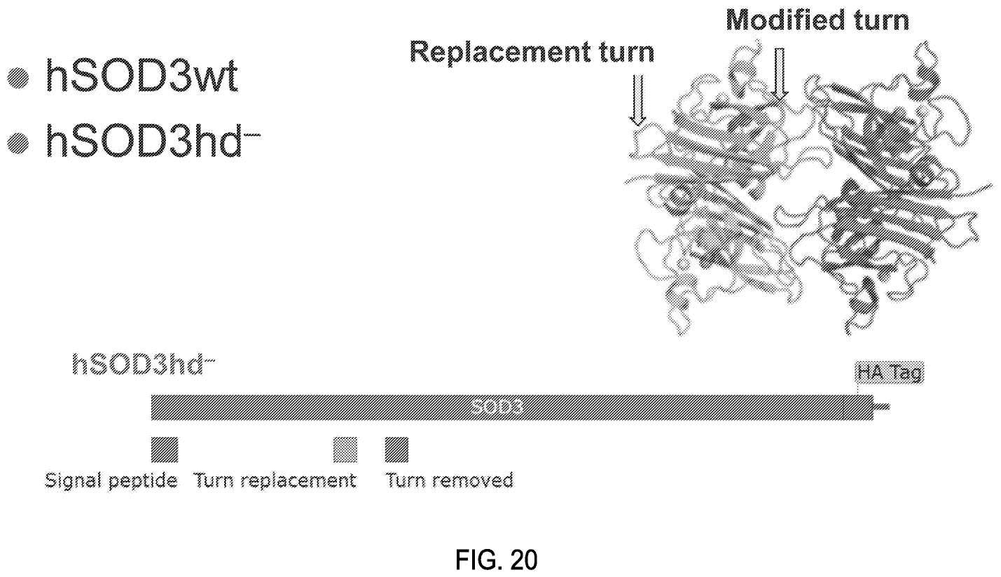

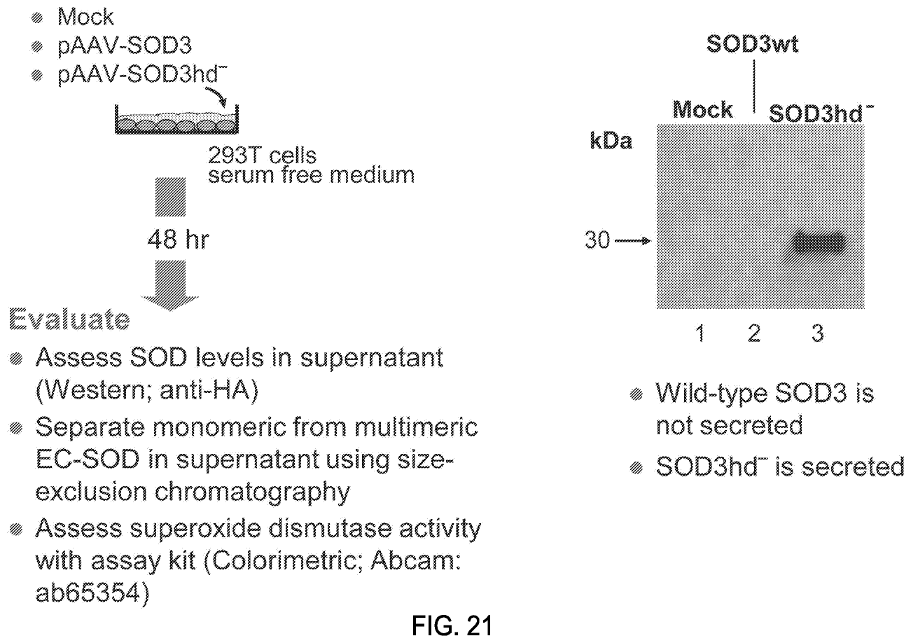

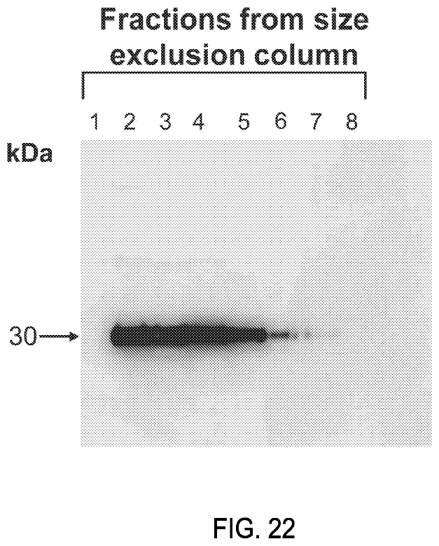

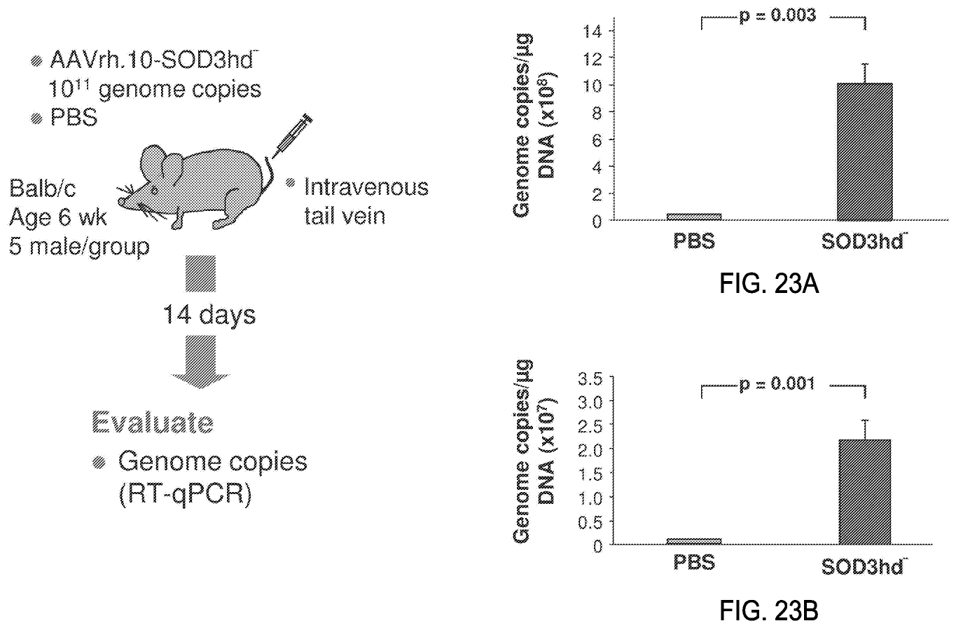

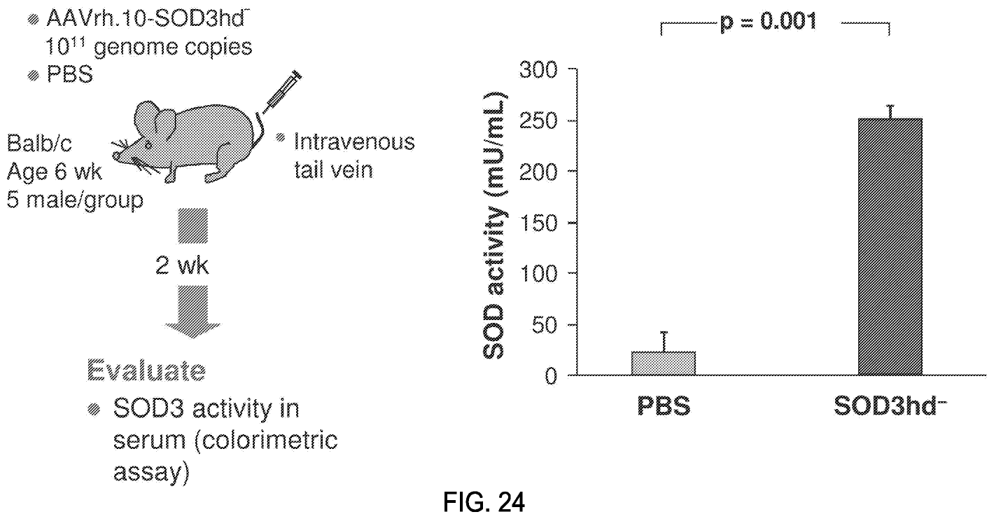

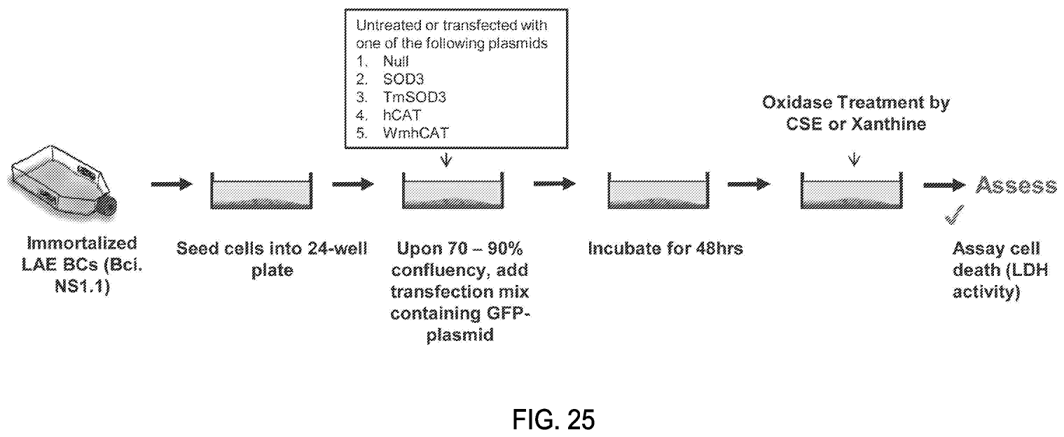

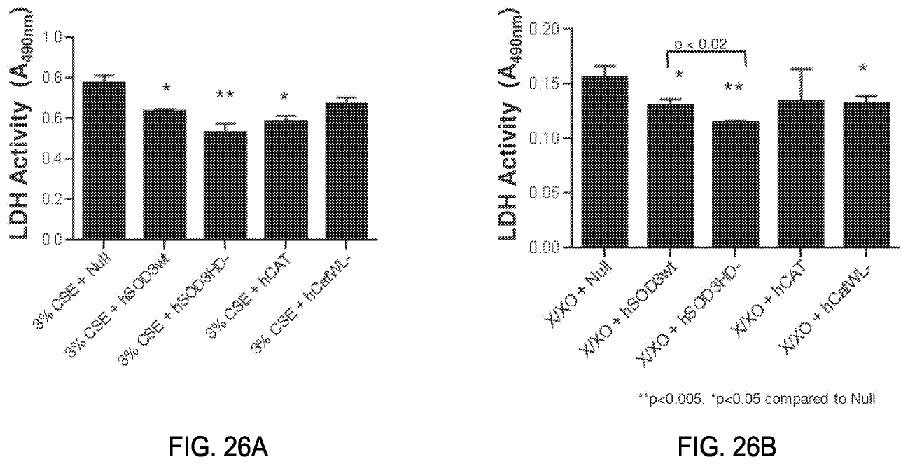

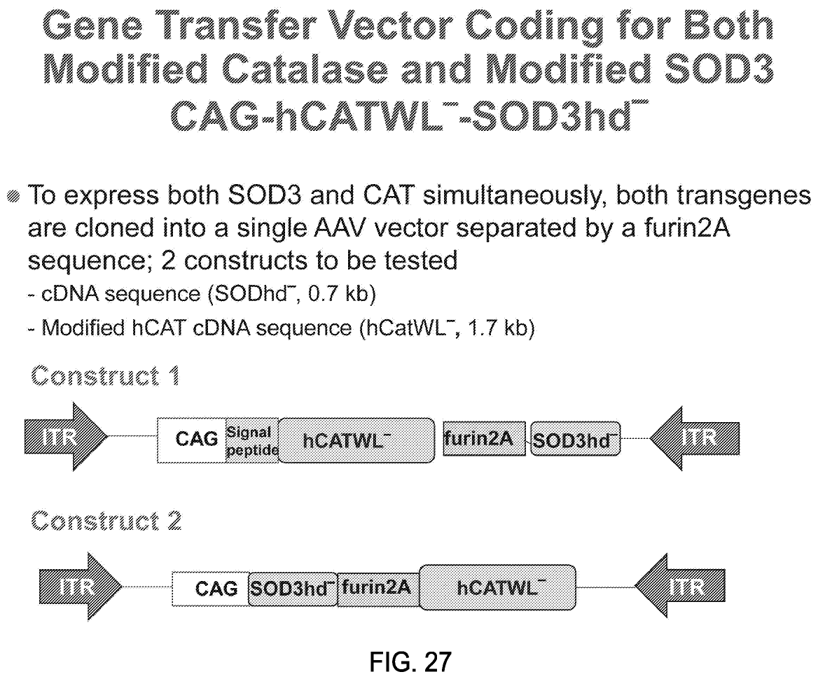

Three modifications of the human catalase sequence were assessed: hCatNT⁻, hCatWL⁻ and hCatNT⁻WL⁻. Assessment of the culture supernatant after transfection of these plasmids into 293T cells in serum free mediate demonstrated, all 3 were secreted (FIG. 5A). However, of the 3 constructs, only the wrapping loop domain deletion (hCatWD⁻) retained catalase activity (FIG. 5B). Bis-Tris gel analysis demonstrated hCAThd⁻ was secreted as a monomer (FIG. 5C). Based on this data, AAVrh.10hCatWD⁻ (LEX 5a) was created. Intravenous administration to mice led to detection of hCatWD⁻ DNA in the liver and lung (typical for AAVrh.10 vectors) (Chiuchiolo et al., 2013), and importantly, easily detectable human catalase activity in serum, e.g., LEX 5a generates a secreted, functional catalase monomer (FIG. 7A-C).

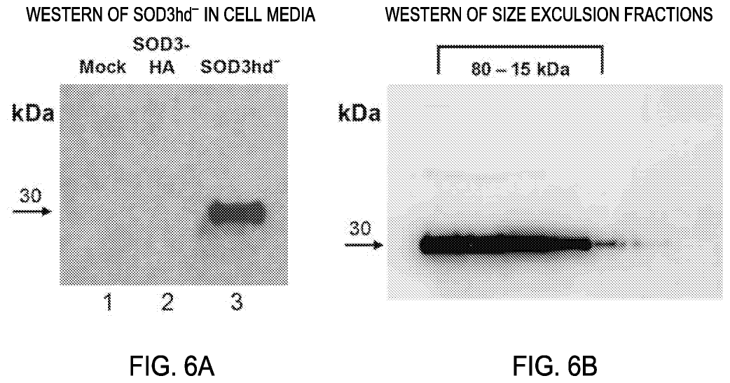

Two modifications were made in a single SOD3 variant (hSOD3hd⁻), including a deletion of residues 50-59 replaced with an in frame copy of the residue segment from 74-80, and a deletion of residues 212-240, the heparin-binding domain. Quantification of the culture supernatant after transfection of this plasmid into 293T cells demonstrated that the hSOD3hd⁻ variant was easily detected in the supernatant (FIG. 6A) and was a monomer (FIG. 6B). Based on this data, AAVrh.10hSOD3hd (LEX 5b) was generated (FIG. 4). Intravenous administration to Balb/c mice led to vector DNA in liver and lung (liver 50-fold higher) and SOD activity in serum (FIG. 7D-F). LEX 5c, LEX 5d. Based on the in vitro and in vivo functional data of LEX 5a and LEX 5b, LEX 5c and LEX 5d were generated to express both catalase and SOD3 modified monomers. Based on expressing 2 genes in 1 AAV construct (De et al., 2008; Wang et al., 2010; Watanabe et al., 2010; Mao et al., 2011; Rosenberg et al., 2012; Hicks et al., 2012; Xie et al., 2014; Hicks et al., 2015; Pagovich et al., 2016; Liu et al., 2016), both LEX 5a and LEX 5b use a single promoter, with the 2 modified cDNA separated by a furin 2A cleavage site which directs cleavage of the resulting precursor protein to generate the 2 function proteins. Two constructs are tested: LEX 5c where the hCatWD⁻ sequence proceeds the hSOD3hd⁻ sequence, and LEX 5d where the 2 cDNA coding sequences are flipped. Both will be tested for in vitro and in vivo function.

Experimental design. The studies focus on comparing LEX 5a, LEX 5b, LEX 5c, and LEX 5d in vitro and in vivo. The following criteria are used to rank the candidates for the same vector dose: (1) in vitro—levels of secreted functional antioxidant against 02, H₂O₂ oxidant stress; (2) in vivo—persistent levels of functional antioxidants in serum and lung epithelial lining fluid.

TABLE I
In Vitro Comparison of the 4 Candidate Expression Plasmids

| Expression plasmids¹ | Parameters² | Rank³ |
|---|---|---|
| hCatWD⁻, hSOD3hd⁻, | Catalase level and activity; | 1-5 for each |
| hCatWD⁻/hSOD3hd⁻, | inhibition of H₂O₂ challenge; | parameter |
| hSOD3hd⁻/hCatWD⁻, | SOD level and activity; | |
| Control | inhibition of O₂⁻ challenge | |

¹Plasmids (4 μg) will be transfected with PEI into 293T cells in serum free media. After 72 hr, the media is collected and assessed. “Control” - plasmid with no transgene. All studies are carried out in quadruplicate with each plasmid assessed in quadruplicate.
²Levels of human catalase and SOD3 are tested by ELISA; catalase activity by colorimetric assay (Thermo Fisher), SOD3 activity by colorimetric assay (Abcam); and H₂O₂ and O₂⁻ challenge to human pulmonary microvascular endothelium and to human airway epithelium).
³Each assay is ranked from 1 (worst, no effect) to 5 (best, averaging the 4trials). The rankings are added for a total score from 6 (worst) little or to 30 (best).

To compare in vitro the levels of expression of secreted, functional modified catalase and/or SOD3, mediated by the expression cassette of the 4 AAVrh.10 antioxidant vectors (hCatWL⁻, hSOD3hd⁻, hCatWL⁻/hSOD3hd⁻ and hSOD3hd⁻/hCatWL⁻), were used The data demonstrates that both hCatWD⁻ (expression cassette for LEX 5a) and hSOD3hd⁻ (expression cassette for LEX 5b) generate monomers and both function in vitro and in vivo to generate catalase and SOD, respectively. The goal of aim 1 is to carry out comparative testing of the 4 expression cassettes by transfecting plasmids (hCatWD⁻, hSOD2hd⁻, hCatWD⁻/hSOD3hd⁻ and hSOD3hd⁻/hCatWD⁻) into 293T cells in serum free media as a function of dose. After 72 hours, the resulting supernatant are tested for: (1) catalase and SOD level; (2) catalase and SOD activity; and (3) ability of the supernatant to protect human microvascular endothelium and human airway epithelium from oxidant stress (O₂⁺ and H₂O₂). The in vitro assessments of the 4 plasmids will be ranked as detailed in Table 11.

TABLE II
In Vivo Comparisons of the 4 Candidate Vectors

| | | | Time points | |
|---|---|---|---|---|
| Vectors¹ | Dose² | Parameters³ | (wk)⁴ | Rank |
| LEX 5a, | 10⁹, | Serum, ELF catalase | 0, 2, 4, 12 | 1-5 for |
| b, c, d, | 10¹⁰, | level, activity and | | each |
| and | 10¹¹ | inhibition of H₂O₂ | | parameter |
| control | genome | challenge; serum, ELF | | |
| | copies | SOD level, activity | | |
| | | and inhibition of O₂⁻ | | |
| | | challenge | | |

^{1 1}control - identical to the other vectors, but with no transgene; n = 5 male and n = 5 female/group/time point.
²Intravenous administration.
³Similar to Table I, footnote ², with serum or lung epithelial lining fluid (ELF) as a function of amount is substituted for the supernatants.
⁴Assessment of liver, lung, serum and ELF at 0, 2, 4, and 12 wk.
⁴As described in Statistics, the total ranking is a combination of the in vitro and in vivo assessment, with the in vivo ranking worth 2-fold that of the in vitro ranking.

To quantify in vivo the ability of the 4 AAVrh.10 antioxidant vectors to express secreted, functional modified catalase and/or SOD3 capable of protecting lung endothelium and epithelium from O₂⁺ and H₂O, stress. The goal is to compare the LEX 5a, b, c and d vectors in vivo. From prior experience with dual expression cassettes in AAVrh.10 vectors (De et al., 2008; Wang et al., 2010; Watanabe et al., 2010; Mao et al., 2011; Rosenberg et al., 2012; Hicks et al., 2012; Xie et al., 2014; Hicks et al., 2015; Pagovich et al., 2016; Liu et al., 2016), since LEX 5a and LEX 5b are functional, there is no reason why the designs of LEX 5c and LEX 5d will not be functional, although there may be differences in the relative expression of the catalase vs SOD3 in the expression cassettes of LEX 5c vs LEX 5d. To compare the 4 vectors, the same doses 10⁹, 10¹⁰, and 10¹¹ genome copies) of intravenous administration are compared in Balb/c male and female mice. At 0, 2 weeks, 1 month and 3 months, the following parameters will be assessed: (1) Liver and lung vector DNA; (2) Liver and lung expression cassette mRNA; (3) catalase and SOD levels and activity in serum and lung epithelial lining fluid (ELF); and (4) serum of the treated mice will be tested for protection of human microvascular endothelium against the stress of O₂⁺ and H₂O₂ in vitro and ELF tested for protection of human airway epithelium against the same oxidant stress. The 3 month time-point will be the last time-point based on the extensive data that, if AAVrh.10-mediated expression of secreted proteins remain stable at 3 months, they will remain stable for the life of the animal (Chiuchiolo et al., 2013; De et al., 2008; De et al., 2006) The choice of Balb/c mice is based on the expression that this strain tolerates human protein expression in vivo without generating immunity against the human protein (Rosenberg et al., 2012). If there are any immunity issues (noted by loss of expression), we will switch the mouse strain to C57Bl/6 which also tolerate human cell genes expressed by AAV vectors (De et al., 2006). The in vivo assessments are ranked as detailed in Table II. The in vitro and in vivo rankings are combined to get an overall ranking.

The plasmid expression cassettes are ranked by 6 efficacy assays; the same assays are used in aim 2 to assess serum and lung ELF.

Catalase levels. ELISA (Abcam).

SOD3 levels. ELISA (Biocompare).

Catalase activity. Colorimetric assay (Thermo Fischer).

SOD3 activity. Colormetric assay (Abeam).

Endothelium challenge with O₂⁺ and H₂O₂. Human microvascular endothelial cells (Lonza) will be challenged with superoxide (chemically using Fenton reaction) or H₂O₂. At 24 and 48 hours, cell death (lactate dehydrogenase levels in supernatant), oxidant response (quantitative PCR for mRNA levels of oxidative stress genes. For protection, cells will be pretreated with plasmid supernatant, serum or ELF.

Epithelium challenge with O₂⁺ and H₂O₂. The assays are identical to that of the endothelium but with human airway epithelium substituted for the endothelium. The human epithelium will be derived over 28 days from human normal basal cells on type IV collagen with air-liquid interface cultures.

AAVrh.10 vector production. The vector is produced by co-transfection into human embryonic kidney 293T cells (HEK 293T; ATCC) of the expression plasmid together with a plasmid carrying the AAV2 Rep gene, the AAVrh.10 Cap gene for proteins VP1, 2 and 3 (which define the serotype of the produced rh.10 AAV vector) and the adenovirus helper functions of E2, E4 and VA RNA (Collaco et al., 1999; Hicks et al., 2016). The vector is purified by iodixanol gradient and QHP anion exchange chromatography. Vector genome titers will be determined by quantitative TaqMan real-time PCR analysis (Mayginnes et al., 2006)

Animal models. Vectors are intravenously administered at each of 3 doses (10⁹, 10¹⁰, 10¹¹) to the tail vein of Balb/c mice. All studies are done with n=5 male and 5 female mice at each data point. Lung ELF, obtained by fiberoptic bronchoscopy and lavage is a mixture of the saline used to recover the ELF and the actual ELF. The volume of recovered ELF is quantified using the urea method (Rennard et al., 1985) and the level of catalase or SOD3 expressed in μM per ELF volume. Serum by tail vein bleed and lung ELF are assessed at 0 (pre-therapy), 2, 4, 12 weeks. At sacrifice liver and lung are collected from the mice for analysis of vector genome and gene expression and transferred to labeled clean 15 ml conical tube, with 1 ml of RNAlater (Qiagen) per 100 mg tissue for DNA/mRNA isolation and stored at 4° C. overnight. Samples are homogenized at 4° C. for 10-20 minutes and 1 sample used for each DNA and mRNA analysis by real time PCR using a primer probe set specific to the transgene.

### REFERENCES

Statistics NCfH. Health, United States, 2016: With Chartbook on Long-term Trends in Health. Health, United States. Hyattsville (Md.) 2017.
Organization WH. The top 10 causes of death http://www.who.int/mediacentre/factsheets/fs310/en/2017 [
Disease GIfCOL. Global Strategy for the Diagnosis, Management, and Prevention of Chronic Obstructive Pulmonary Disease. http://goldcopd.org/wp-content/uploads/2017/11/GOLD-2018-v6.0-FINAL-revised-20-Nov_WMS.pdf2018.
Antonvuk et al., J. Mol. Biol., 388:310 (2009).
Bell et al., Am. Rev. Respir. Dis., 124:72 (1981).
Benton et al., Respirology (2018).
Casas et al., Antioxid. Redox Signal, 23:1171 (2015).
Chance, Acta Chemica. Scandina. 1., 1:236 (1947).
Chen & Tang, Exp. Eye Res., 93:599 (2011).
Chiuchiolo et al., Hum. Gene Ther. Clin. Dev., 24:161 (2013).
Chu et al., Circ. Res., 92:461 (2003).
Collaco et al., Gene. 23:397 (1999).
Danel et al., Hum. Gene Ther., 2:1487 (1998).
Davidoff et al., Blood, 102:480 (2003).
Davies, Biochimie., 83:301 (2001).
De B P et al., Mol. Ther., 16:203 (2008).
De et al., Mol. Ther., 13:67 (2006).
Devasagayam & Kamat, Indian J. Exp. Biol., 40:680 (2002).
Elmasry et al., Egyptian Journal of Chest Diseases and Tuberculosis, 64:813 (2015).
Erzurum et al., J. Appl. Physiol., 75:1256 (1993).
Foronjy et al., Pulm. Pharmacol. Ther., 21:370 (2008).
Foust et al., Mo. Ther., 21:2148 (2013).
Fukai & Ushio-Fukai, Antioxid. Redox Signal., 1:1583 (2011).
Gao et al., Respirology, 20:722 (2015).
Goyal & Basak, Protein Cell., 1:888 (2010).
Griess et al., Free Radic. Biol. Med., 112:464 (2017).
Guenzel et al., Hum. Gene Ther., 26:153 (2015).
Guinness & Sapey, J. Clin. Med., (2017).
Hicks et al., Cancer Gene Ther., 22:1 (2015).
Hicks et al., PLoS One, 11:e0162978 (2016).
Hicks et al., Sci. Transl. Med., 4:140 (2012).
Hubbard & Crystal, Respiration., 50 Suppl 1:56 (1986).
Irshad & Chaudhuri, Indian J. Exp. Biol., 40:1233 (2002).
Janssen et al., Lab Invest., 2:261 (1993).
Ko et al., Acta. Crystallogr. D. Biol. Crystallogr., 6:241 (2000).
Li et al., Circulation, 103:1893 (2001).
Lin & Thomas, COPD, 7:291 (2010).
Liu et al., J. Neurosci., 36:12425 (2016).
MacNee, Chest., 117:303S (2000).
Mao et al., Hum. Gene Ther., 22:1525 (2011).
Mayginnes et al., J. Virol. Methods, 137:193 (2006).
Miyazaki et al., Gene, 79:269 (1989).
Niwa et al., Gene, 108:193 (1991).
Olsen et al., J. Biol. Chem., 279:22152 (2004).
O'Reilly. Am. J. Physiol. Lung Cell Mol. Physiol., 281:L291 (2001).
Pagovich et al., J. Allergy Clin. Immunol., 138:1652 (2016).
Paneda et al., Hum. Gene Ther., 20:908 (2009).
Perry et al., Biochimica et Biophysica Acta (BBA)—Proteins and Proteomics, 104:245 (2010).
Pfeifer et al., Gene Ther., 18:1034 (2011).
Pham-Huy et al., Int. J. Biomed. Sci., 4:89 (2008).
Polverino et al., Pulm. Circ., 8:2045894018758528 (2018).
Prasad et al., J. Gene. Med., 13:333 (2011).
Rahman et al., Eur. J. Pharmacol., 533:222 (2006).
Rahman, Biochim. Biophys. Acta., 1822:714 (2012).
Rahman, Cell Biochem. Biophys., 41:167 (2005).
Rahman, Ther. Adv. Respir. Dis., 2:351 (2008).
Rennard et al., J. Appl. Physiol., 60:532 (1985).
Reynolds et al., J. Clin. Invest., 5:165 (1977).
Rosenberg et al., Hum. Gene Ther., 23:451 (2012).
Safo et al., Acta. Crystallogr. D. Biol. Crystallogr., 57:1 (2001).
Sandstrom et al., Biochem. J., 294 (Pt 3):853 (1993).
Sepasi et al., Progress in Biophysics and Molecular Biology (2018).
Shapiro & Ingenito, Am. J. Respir. Cell. Mol. Biol., 32:367 (2005).
Shaykhiev & Crystal, Ann. Am. Thorac. Soc., 11 Suppl., 5:5252 (2014).
Sies et al., Annu. Rev. Biochem., 85:715 (2017).
Sun et al., Mol. Ther., 11:57 (2005).
Vos et al., Lancet., 380:2163 (2012).
Wang et al., Cancer Gene Ther., 17:559 (2010).
Watanabe et al., Gene Ther., 17:1042 (2010).
Woodruff et al., Lancet. 385:1789 (2015).
Xie et al., Gynecol. Oncol., 135:325 (2014).
Yoo et al., J. Clin. Invest., 21:297 (1994).
Yoshida & Tuder. Physiological Reviews. 87:1047 (2007).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A gene therapy vector comprising an expression cassette comprising a nucleic acid sequence coding for a modified catalase that has catalase activity but does not form tetramers or a gene therapy vector comprising an expression cassette comprising a nucleic acid sequence coding for a modified superoxide dismutase that is secreted but does not bind to cell surfaces or does not form tetramers.

2. The gene therapy vector of claim 1 comprising the nucleic acid sequence coding for a modified catalase and a nucleic acid sequence coding for a modified superoxide dismutase that is secreted but does not bind to cell surfaces.

3-6. (canceled)

7. The gene therapy vector of claim 1 wherein the modified catalase has a deletion in the N-terminus in the threading arm domain, which deletion may be of 1 to 80 or any integer between 1 and 80, or wherein the modified catalase has a deletion in the wrapping loop domain, which deletion may be 1, 2, 3, 4, 5, 6, 7, 8, 9, 10, 11, 12, 13, 14, 15, 16, 17, 18, 19, 20, 21, 22, 23, 24, or 25 residues, or wherein the modified catalase has a deletion in the threading arm and the wrapping loop domains, or wherein the modified catalase has a secretory sequence.

8-10. (canceled)

11. The gene therapy vector of claim 1 wherein the modified superoxide dismutase has a deletion in the heparin binding domain, which deletion may be of 1 to 15 or 20 or 25 or more residues, or wherein the modified superoxide dismutase has a replacement of one or more residues of a turn or loop domain, or wherein the modified superoxide dismutase has a deletion and an insertion of one or more residues of a turn or loop domain, or does not bind to heparin.

12-13. (canceled)

14. The gene therapy vector of claim 1 which is a viral vector.

15. The gene therapy vector of claim 14 which is an adenovirus, adeno-associated virus (AAV), retrovirus or lentivirus vector.

16-19. (canceled)

20. The gene therapy vector of claim 2 wherein one vector comprises an expression cassette comprising a nucleic acid sequence coding for the modified catalase and the modified superoxide dismutase, which catalase sequence and superoxide dismutase sequence are separated by a protease substrate sequence.

21. The gene therapy vector of claim 20 wherein the modified catalase is N-terminal to the modified superoxide dismutase

22. (canceled)

23. The gene therapy vector of claim 2 wherein one vector comprises an expression cassette comprising a nucleic acid sequence coding for the modified catalase and another vector comprises an expression cassette comprising a nucleic acid sequence coding for the modified superoxide dismutase.

24. A pharmaceutical composition comprising an amount of the vector of claim 1.

25-26. (canceled)

27. The pharmaceutical composition of claim 24 wherein the vector is an adenovirus, adeno-associated virus (AAV), retrovirus or lentivirus vector.

28-29. (canceled)

30. The pharmaceutical composition of claim 27 wherein the AAV vector is pseudotyped with AAVrh.10, AAV8, AAV9, AAV5, AAVhu.37, AAVhu.20, AAVhu.43, AAVhu.8, AAVhu.2, or AAV7 capsid.

31-32. (canceled)

33. The pharmaceutical composition of claim 27 wherein the amount of the vector is about 1×10¹² to about 1×10¹⁵ genome copies, about 1×10¹¹ to about 1×10¹³ genome copies, or about 1×10¹³ to about 1×10¹⁵ genome copies.

34-35. (canceled)

36. A method to prevent, inhibit or treat oxidative damage, or prevent, inhibit or treat COPD, respiratory distress syndrome or fibrotic interstitial lung disease, in a mammal, comprising: administering to the mammal, an effective amount of the vector of claim 1.

37. The method of claim 36 wherein the mammal has or is at risk of having atherosclerosis, cancer, diabetes, rheumatoid arthritis, post-ischemic perfusion injury, myocardial infarction, cardiovascular diseases, chronic inflammation, stroke, septic shock, or other degenerative and neurological diseases such as Alzheimer's disease or Parkinson's disease.

38. (canceled)

39. The method of claim 36 wherein the mammal is a human.

40. The method of claim 36 wherein an amount of a viral vector encoding the modified catalase and an amount of a viral vector encoding the modified superoxide dismutase is administered.

41. The method of claim 40 wherein the viral vectors are administered sequentially.

42. The method of claim 40 wherein the viral vectors are administered concurrently.

43. The method of claim 36 wherein a viral vector encoding the modified catalase and the modified superoxide dismutase is administered.
